(19) United States
(12) Patent Application Publication
(10) Pub. No.: US 2020/0237878 A1
(43) Pub. Date: Jul. 30, 2020

(54) COMPOSITIONS AND METHODS FOR TREATING PSEUDOANEURYSM

(71) Applicant: Oxford University Innovation Limited, Oxford (GB)

(72) Inventors: Regent LEE, Oxford (Oxfordshire) (GB); Ashok HANDA, Oxford (Oxfordshire) (GB); Eleanor STRIDE, Oxford (Oxfordshire) (GB)

(21) Appl. No.: 16/652,486

(22) PCT Filed: Sep. 24, 2018
(86) PCT No.: PCT/GB2018/052710

§ 371 Date: Mar. 31, 2020

(30) Foreign Application Priority Data
Oct. 5, 2017 (GB) 1716239.7

(51) Int. Cl. A61K 38/36; A61K 9/00; A61K 9/50; A61K 41/00; A61K 9/51

(52) U.S. Cl. CPC A61K 38/366; A61K 9/0019; A61K 9/0009; B82Y 5/00; A61K 41/0052; A61K 9/5192; A61K 9/5094

(57) ABSTRACT

A composition for use in the treatment of a pseudo aneurysm in a subject, said composition comprising: i) microbubbles; ii) a magnetic material; and iii) a blood clotting agent; said treatment comprising administering said composition directly into the pseudo aneurysm and applying a magnetic field to the pseudo aneurysm so as to retain the blood clotting agent within the pseudo aneurysm for a prolonged time period compared to a case where no magnetic field is applied to the pseudo aneurysm.

The present invention relates to compositions for use in methods of treating pseudo aneurysms, as well as to methods of treating pseudo aneurysms. The invention also relates to methods of imaging a pseudo aneurysm and compositions and devices for use in said methods.

A pseudo aneurysm (PSA), or false aneurysm, occurs when blood escapes from the lumen of an artery through a defect in one or more layers of the arterial wall and forms a localized pocket of flow either beneath the adventitia (outer wall of the artery) or in the surrounding tissues. PSA is a recognised complication from catheterisation of femoral artery, with the reported incidence of up to 8% after therapeutic endovascular procedures. With the increasing application of therapeutic endovascular procedures, PSAs are becoming a common problem in clinical practice.

Symptoms of a PSA can include pain, swelling, bruising, and free extravasation of blood (“rupture”) into the surrounding tissue. Traditional methods of treatment include open surgical repair, or ultrasound guided compression of the PSA. Surgical repair of PSA can be complicated by infection of the wound, prolonged hospital stay for aftercare, and the requirement of general anaesthesia. The latter is a particular challenge, as patients who are subject to endovascular interventions typically have higher medical co-morbidities that render them unsuitable for the open surgery approach (coronary, peripheral arterial) in the first place. Ultrasound guided mechanical compression is poorly tolerated as the area affected is already swollen and tender.

One medicament known for treating PSA is Thrombin. Thrombin induces thrombosis by converting fibrinogen to fibrin, which forms the scaffold for thrombus (clot). The off label use of direct injection of thrombin as a treatment for PSA has been in practice since the late 1990s. Ultrasound guided thrombin injection (UGTI) subsequently became a mainstream therapeutic option, followed by the endorsement by NICE as a treatment option for PSA in 2004. A recent Cochrane review also concluded UGTI as an effective treatment for femoral artery PSAs.

However, UGTI is associated with inadvertent intra-arterial injection of thrombin resulting in complications such as acute intra-arterial thrombosis or distal arterial embolization. As such, the application of UGTI is limited to PSAs with “favourable” anatomy such as a long and narrow neck. It is also routine practice to limit the amount of thrombin injected during each treatment episode in order to minimise the potential intra-arterial “spill over” of thrombin. Failure of UGTI due to incomplete thrombosis or unfavourable anatomy would lead to the need of open surgical repair, which can be further fraught with potential complications.

Further, when treating a PSA using conventional UGTI, it is difficult for the physician to know whether the treatment has been effective at the time of treatment. It is only known at a post treatment check-up or if the patient returns with continued symptoms.

It is an aim of the present invention to at least partially address the problems above.

The present invention provides a composition for use in the treatment of a pseudo aneurysm in a subject, said composition comprising

i) microbubbles;

ii) a magnetic material; and

iii) a blood clotting agent.

The present invention also provides a composition for use in the treatment of a pseudo aneurysm in a subject, said composition comprising

i) microbubbles;

ii) a magnetic material; and

iii) a blood clotting agent;

said treatment comprising administering said composition directly into the pseudo aneurysm and applying a magnetic field to the pseudo aneurysm so as to retain the blood clotting agent within the pseudo aneurysm for a prolonged time period compared to a case where no magnetic field is applied to the pseudo aneurysm.

The invention also provides a method of imaging a pseudo aneurysm comprising:

applying a magnetic field to the pseudo aneurysm so as to retain within the pseudo aneurysm for a prolonged time period compared to a case where no magnetic field is applied to the pseudo aneurysm microbubbles comprising a magnetic material and a blood clotting agent that have been pre-administered directly into the pseudo aneurysm; imaging the pseudo aneurysms using ultrasound, the microbubbles providing a contrast agent.

The invention further provides a method for treating a pseudo aneurysm in a subject, said method comprising administering to said subject a composition of the invention, said method comprising administering said composition directly into the pseudo aneurysm and applying a magnetic field to the pseudo aneurysm so as to retain the blood clotting agent within the pseudo aneurysm for a prolonged time period compared to a case where no magnetic field is applied to the pseudo aneurysm.

The invention further provides a composition of the invention for use in the manufacture of a medicament for the treatment of a pseudo aneurysm in a subject, said treatment comprising administering said medicament directly into the pseudo aneurysm and applying a magnetic field to the pseudo aneurysm so as to retain the blood clotting agent within the pseudo aneurysm for a prolonged time period compared to a case where no magnetic field is applied to the pseudo aneurysm.

The invention also provides a composition comprising i) microbubbles; ii) a magnetic material; and iii) a blood clotting agent. The microbubbles, the magnetic material and/or the blood clotting agent are preferably as described in more detail herein.

Further features and advantages of the present invention will be described below by way of non-limiting examples and by reference to the accompanying drawings in which:

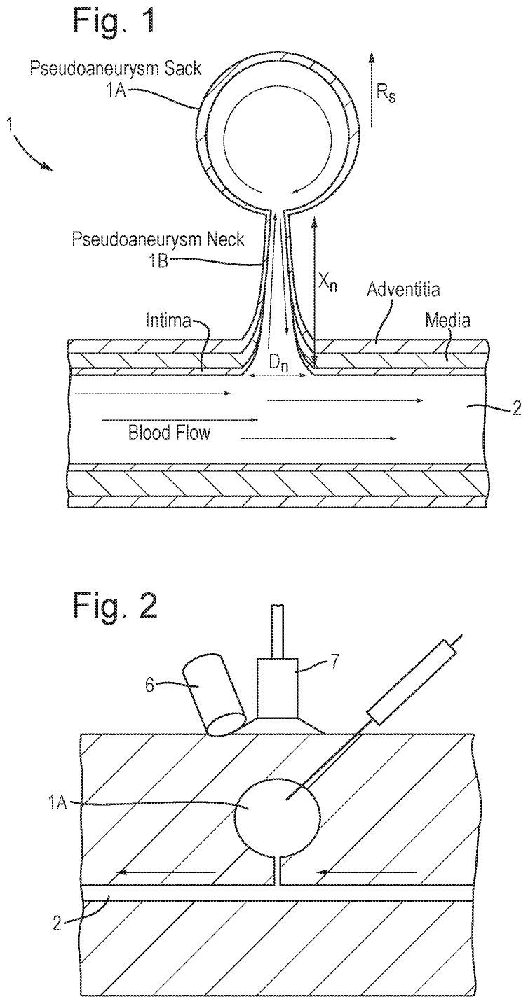

FIG. 1 is a schematic diagram of pseudo aneurysm; there are three main parameters associated with the aneurysm that can potentially affect the outcome of the microbubble treatment. Rₛ is the radius of the sack, χₙ the length of the neck and Dₙ the diameter of the neck.

FIG. 2 is a schematic diagram of apparatus for carrying out a method in accordance with the invention.

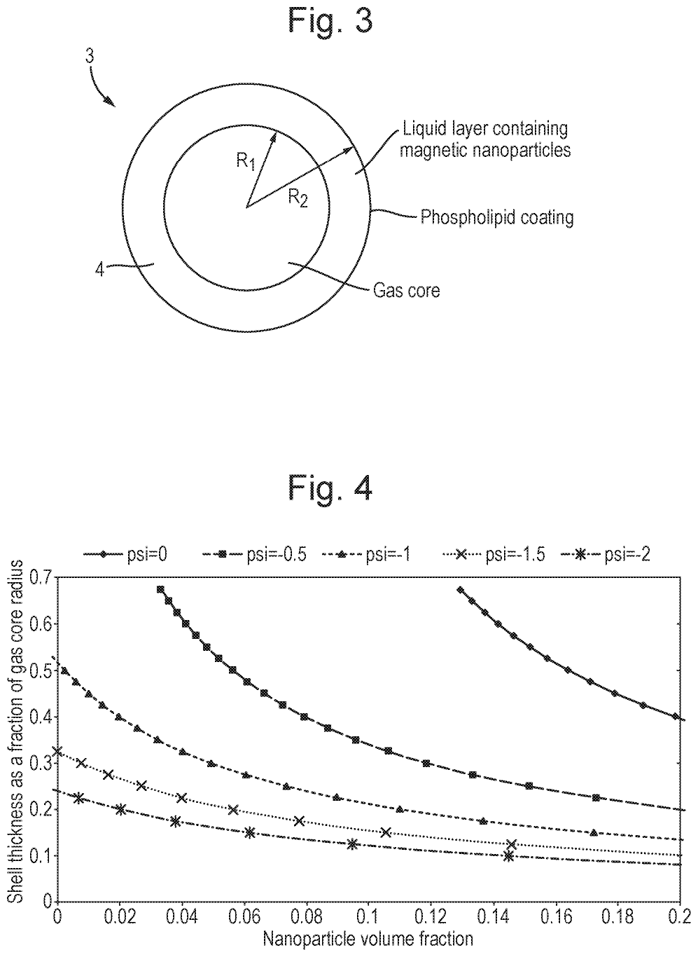

FIG. 3 shows a schematic cross-section of a magnetic microbubble.

FIG. 4 is a graph showing different values of ψ (ψ is 0, −0.5, −1, −1.5 or −2), where nanoparticle volume fraction is plotted on the x-axis and shell thickness as a fraction of gas core radius is plotted on the y-axis.

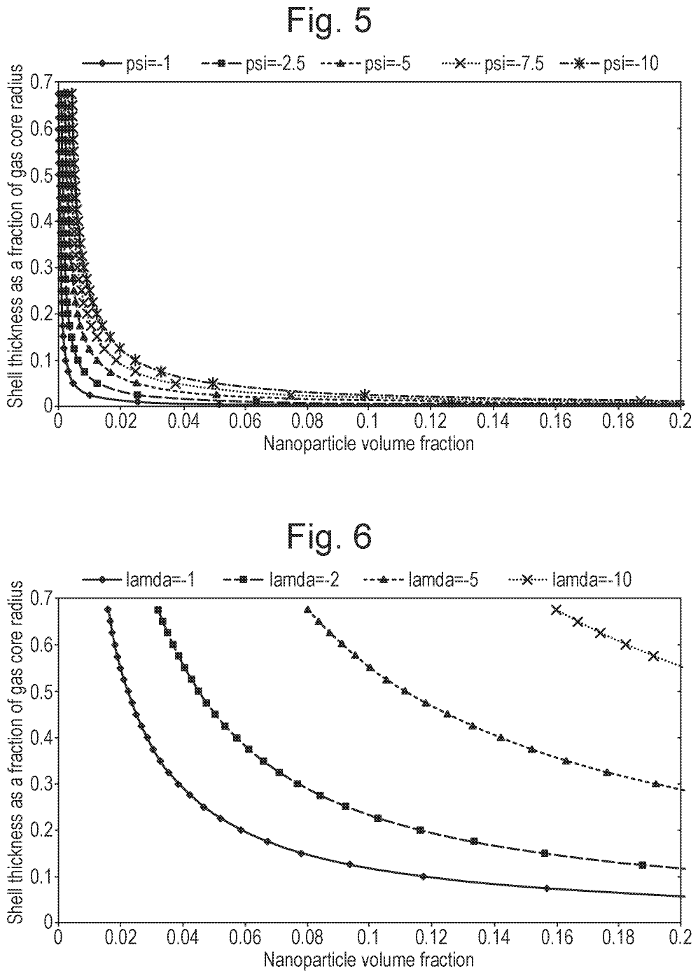

FIG. 5 is a graph showing different values of φ (φ is −1, −2.5, −6, −7.5 or −10) for a given magnetic field ((B. ∇) B=18 T²/m has been used as an example), where nanoparticle volume fraction is plotted on the x-axis and shell thickness as a fraction of gas core radius is plotted on the y-axis.

FIG. 6 is a graph showing different values of λ (λ is −1, −2, −5 or −10) for a given magnetic field and flow velocity ((B. ∇) B=18 T²/m and a flow velocity of 10⁻⁴ m/s have been used as an example), where nanoparticle volume fraction is plotted on the x-axis and shell thickness as a fraction of gas core radius is plotted on the y-axis.

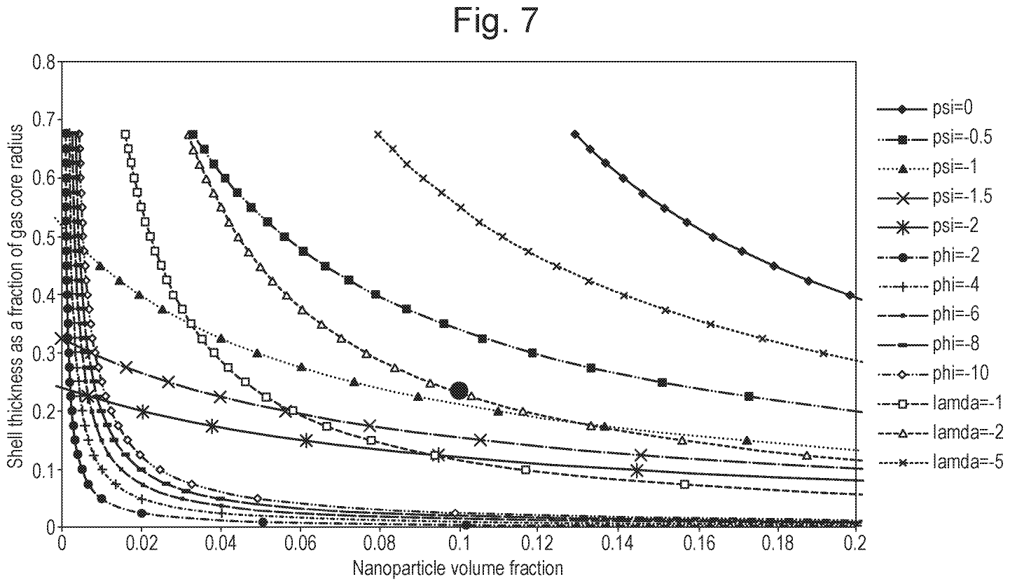

FIG. 7 is a formulation map obtained by combining graphs for ψ, (φ and λ, such as FIGS. 2 to 4, and it shows the range of microbubble designs that satisfy the conditions set out for ψ, φ and λ for ((B. ∇) B 18 T/m and a flow velocity of 10⁻⁴ m/s. An optimised microbubble design is represented by a circle in the figure.

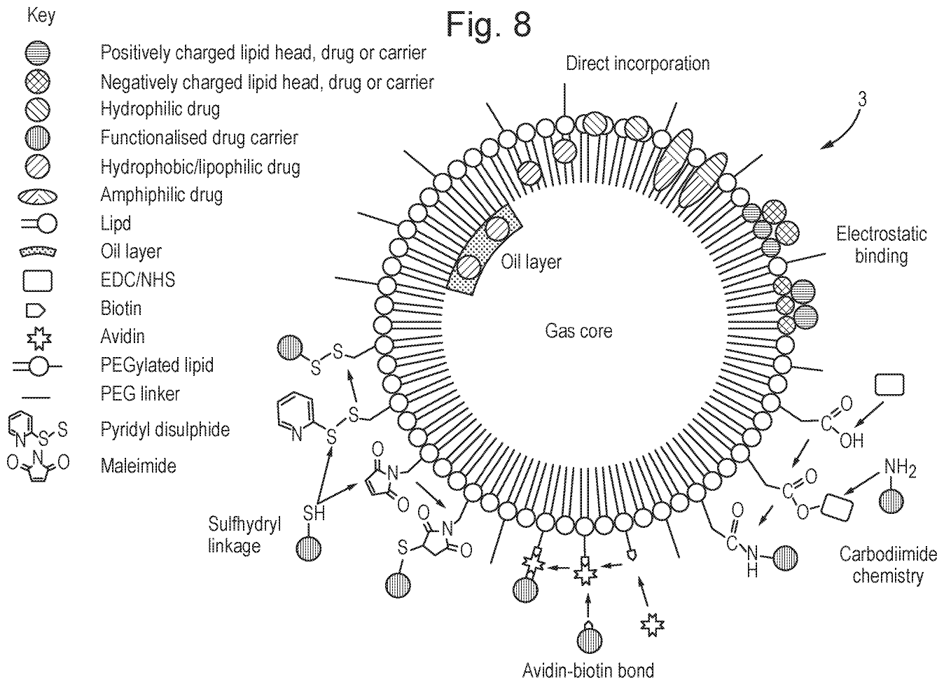

FIG. 8 shows a schematic of different potential loading techniques for lipid microbubbles.

All techniques are applicable to protein and polymer microbubbles. Direct incorporation relies on the hydrophobic (or lipophilic), hydrophilic, or amphiphilic interactions between the drug and shell. Electro-static binding relies on charge differences between the drug or drug carrier and shell molecules. A further method involves loading the drug into an oil layer trapped beneath the shell. Approaches relying on functionalization of the shell and/or drug carrier include carbodiimide chemistry on exposed carboxylic acid (—COOH) or amine (—NH₂) groups, tagging with avidin or biotin to form an avidin-biotin bond and using pyridyl disulphide or maleimide to link to exposed sulfhydryl (—SH) groups.

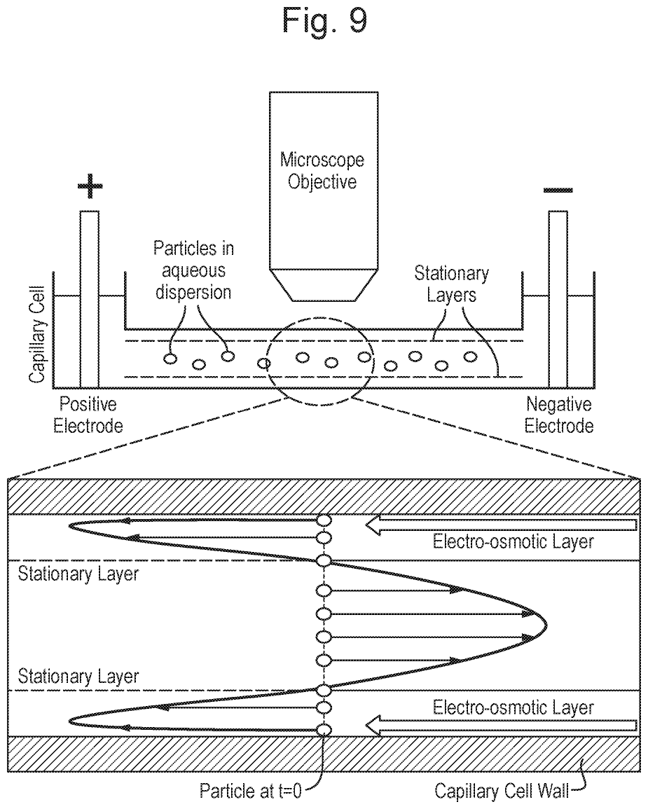

FIG. 9 shows an experimental setup for measuring the zeta potential of microbubbles.

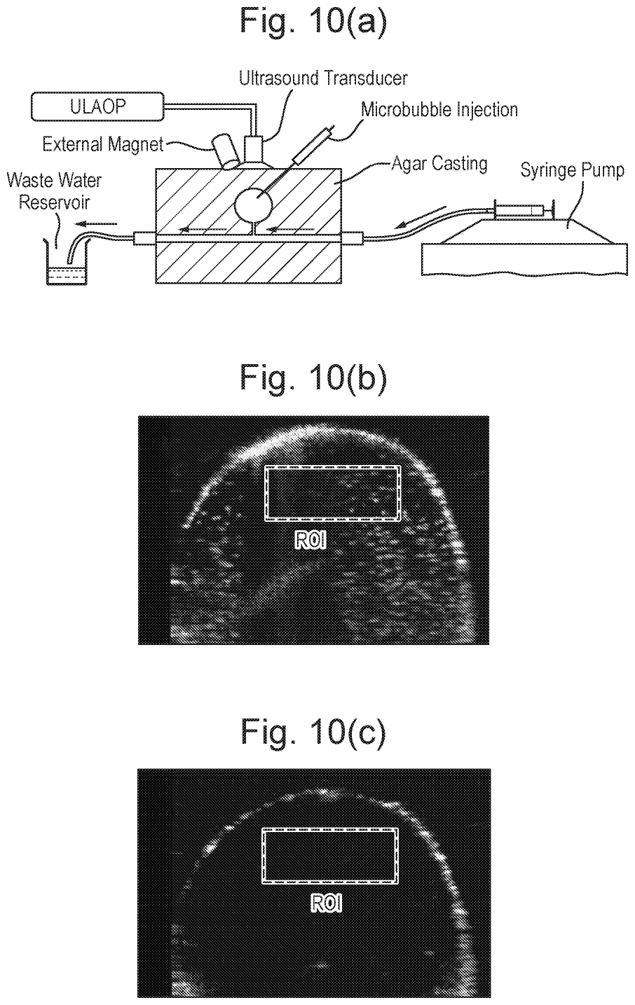

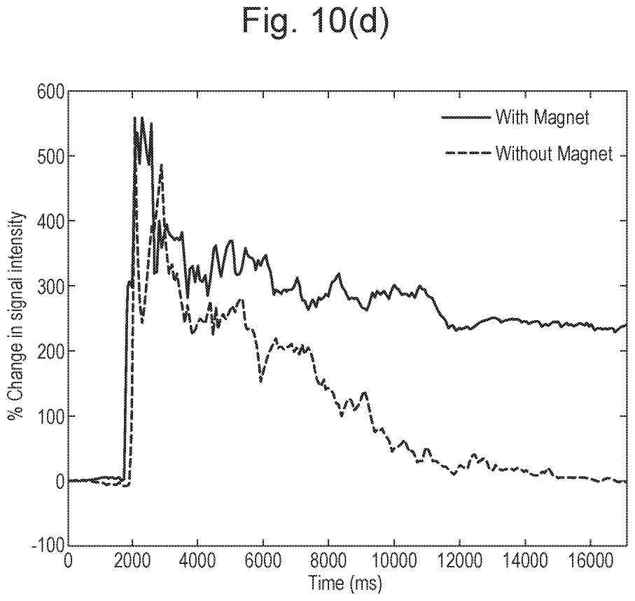

FIG. 10 shows: A) an experimental set up for demonstrating the feasibility of targeted delivery and retention of magnetic microbubbles in a PSA phantom system, comprising a basic model of PSA phantom with pulsatile flow condition, where direct injection of magnetic microbubbles is achieved through a needle; B) microscope images of the phantom PSA with a magnetic present; C) microscope images of the phantom PSA with a magnetic; D) a graph showing signal intensity indicative of the presence of microbubbles in the PSA phantom with and without a magnet.

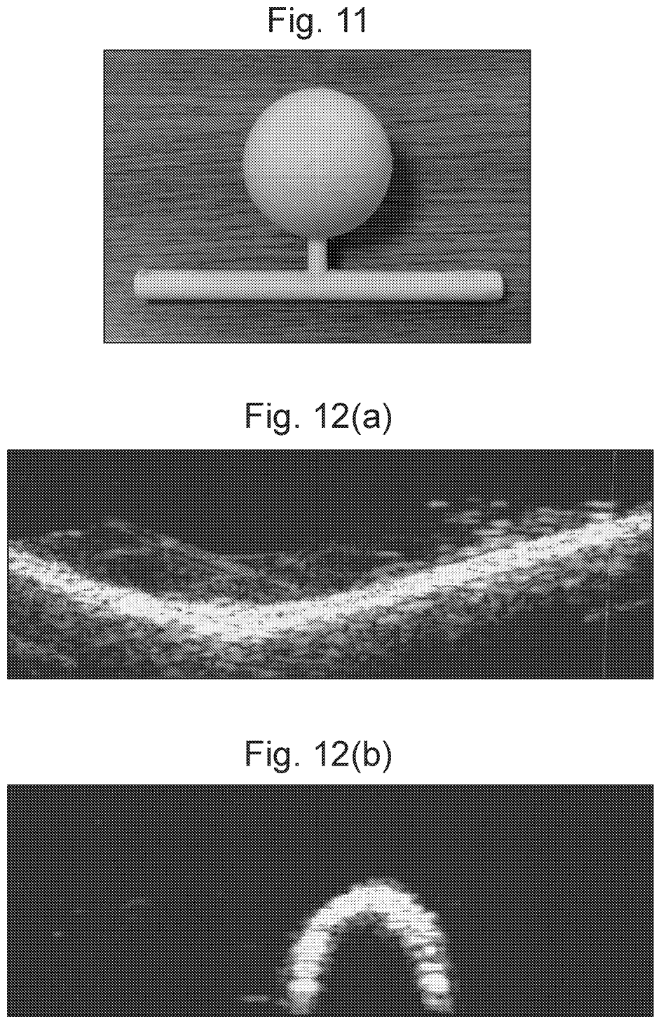

FIG. 11 shows an image taken of a simplified pseudo aneurysm model printed using water soluble PVA filament and later inserted into the US phantom.

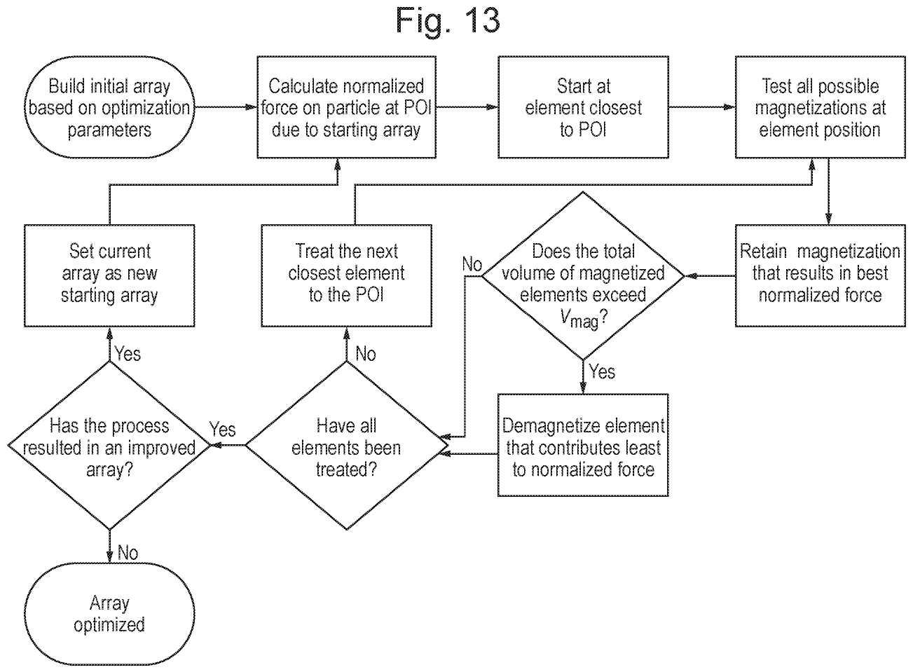

FIG. 12 shows a) an elevation view of the artery structure b) an end elevation view demonstrating the hollow structure of the cylinder. FIG. 13 shows a flow diagram representing the routine for optimizing magnet arrays within an arbitrary parameter space.

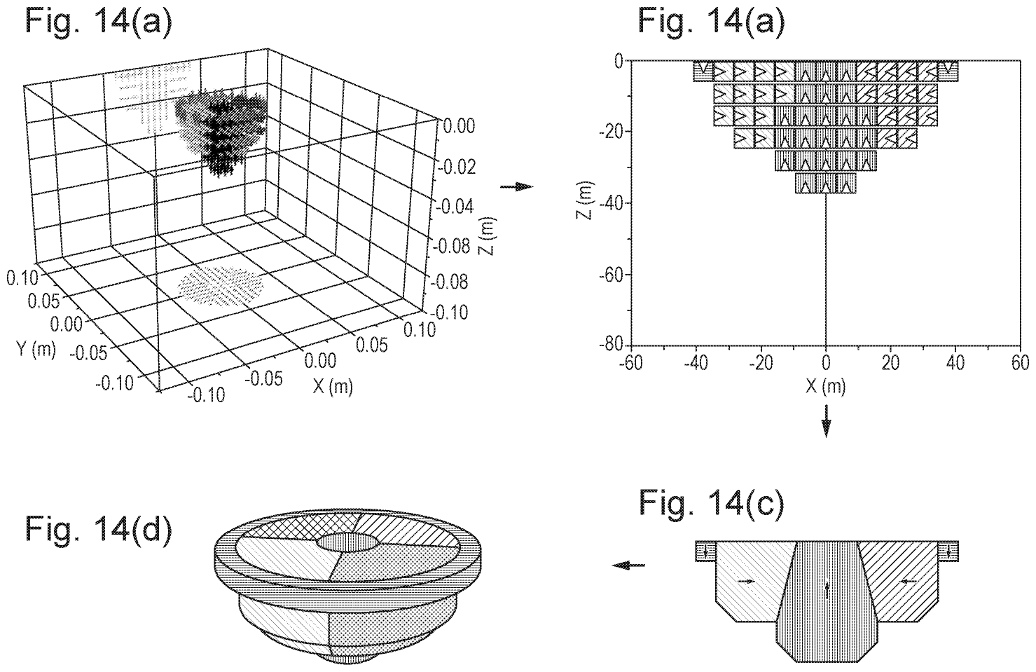

FIG. 14 shows: (a) The result of an optimization is given in terms of an arrangement of magnetization vectors which each represent the final orientation of an element in space. Vectors are colour-coded by magnetization direction. Projections onto the x-y and x-z planes are displayed on the back-planes. (b) Where the output can be approximated by a cylindrically symmetrical arrangement, the optimized configuration is projected onto a 2D plane to generate a 2D vector map of a side cross-section through the middle of the array and (c) regions with the same magnetization are merged into individual shapes. (d) The resultant magnet arrangement can then be specified in terms of a series of cylindrically symmetrical segments with different dimensions.

FIG. 1 is a schematic diagram of a pseudo aneurysm 1. The pseudo aneurysm 1 comprises a sac 1A and a neck 1B. The neck 1B connects the pseudo aneurysm 1 to an artery 2. Blood can flow between the artery 2 and the pseudo aneurysm sac 1A via the neck 1B.

The present invention provides a composition for use in the treatment of pseudo aneurysm (PSA), the composition comprising:

(i) microbubbles (3);

(ii) a magnetic material (4); and

(iii) a blood clotting agent.

The microbubbles, magnetic material and blood clotting agent preferably comprised in the composition are described in more detail herein.

The invention also provides a composition for use in the treatment of a pseudo aneurysm in a subject, said composition comprising

i) microbubbles;

ii) a magnetic material; and

iii) a blood clotting agent;

said treatment comprising administering said composition directly into the pseudo aneurysm and applying a magnetic field to the pseudo aneurysm so as to retain the blood clotting agent within the pseudo aneurysm for a prolonged time period compared to a case where no magnetic field is applied to the pseudo aneurysm.

The composition is useful in the treatment of PSA. As described in more detail herein, the treatment of PSA typically comprises administration of the composition directly into the pseudo aneurysm 1, preferably the sac 1A of the pseudo aneurysm 1. As described herein, any suitable method of administration can be used. Preferably, the composition is administered by injection, e.g. ultrasound guided injection. During an ultrasound guided injection, an ultrasound element 7 is used to image the pseudo aneurysm 1 and the surrounding tissue. The path of a needle through which the injection is made is directed to the pseudo aneurysm 1 based on the ultrasound image. The ultrasound is preferably focused on the pseudo aneurysm 1.

According to a second embodiment, the present invention also provides a method of imaging a pseudo aneurysm 1. In this embodiment, the microbubbles 3 act as a contrast agent for ultrasound imaging performed using an ultrasound element 7. Because the microbubbles comprise the blood clotting agent and the magnetic particles, it is possible to determine the effectiveness of the treatment based on the ultrasound images of the microbubbles within the pseudo aneurysm 1 and the artery 2.

In both the above embodiments, preferably before or simultaneously with the administration of the composition, a magnetic field is applied to the pseudo aneurysm 1 by a magnetic element 6, as shown in FIG. 2. The magnetic field is applied so as to retain the blood clotting agent within the pseudo aneurysm 1 for a prolonged time period compared to a case where no magnetic field is applied to the pseudo aneurysm 1. In this way, the blood clotting agent is retained at the location at which it is administered. Therefore, significant quantities of the blood clotting agent do not leave the pseudo aneurysm and enter the artery, which can cause potentially fatal complications.

Parameters of the magnetic field may be optimised for the treatment of pseudo aneurysms, e.g. based on the location, size and/or shape of the pseudo aneurysm 1. The parameters include the field strength and field gradient of the magnetic field. The location of the pseudo aneurysm 1 may include the depth of the pseudo aneurysm 1 from the skin surface of the patient. The size and/or shape of the pseudo aneurysm 1, may include the volume of the pseudo aneurysm 1, the shape of the sac 1A of the pseudo aneurysm 1 the length of the neck 1B and the diameter of the neck 1B (e.g. maximum, minimum, or average diameter).

The magnetic element 6 may be configured such that the magnitude of the magnetic force exerted on microbubbles 3 varies along an axis extending through the magnetic element 6 (e.g. through one or more bodies of magnetic material forming the magnetic element 6 and has a peak located on the axis at a finite distance from the magnetic element 6. The magnetic element 6 is preferably configured such that the peak force along the axis is located within the target volume of the pseudo aneurysm 1. A sharp rise and fall in the force magnetic force along an axis results in improved retention of microbubbles in line with the axis. There may be a plurality of peaks and the magnetic element 6 may be configured such that any peak is located within the target volume.

However, preferably the maximum peak is located within the pseudo aneurysm 1. The peak magnetic field generated may be from 0.1 T to 9 T. The peak magnetic field gradient generated may be from 1 T/m to 100 T/m.

A magnetic field providing a peak force may be provided by an annular portion of magnetic material forming the magnetic element 6. In this case, the axis on which the peak magnetic force is located coincides with a central axis of the annulus. Alternatively, or additionally, the magnetic material forming the magnetic element 6 may be tapered towards the location of the peak force. The taper in the magnetic material forming the magnetic element 6 may be a continuous taper, or the taper may be stepped, as shown in FIG. 14c. These structures compromise the magnetic force close to the magnetic element 6.

The magnetic material forming the magnetic element 6 may additionally be tapered in a direction away from the location of the peak force in a region of the magnetic material on an opposite side of the magnetic material to the other tapered region.

The body of magnetic material is preferably shaped so as to have a cylindrical portion, a tapered portion at one end of the cylindrical portion (facing the target) and, optionally a tapered portion at the other end of the cylindrical portion.

The magnetic element 6 may be formed from a plurality of bodies of magnetic material having different magnetisation directions (see FIG. 14c). This can provide better control over the magnetic force. For example, the magnetic element 6 may comprise a two different bodies of magnetic material respectively having magnetisation directions having a component in an opposite directions. A body of magnetic material having a magnetisation in one direction may be arranged to surround a body of magnetic material having an opposing magnetisation direction, the bodies being arranged in a part of the magnetic element 6 close to the location of peak force.

A first body of magnetic material is preferably shaped so as to have a cylindrical portion, a tapered portion at one end of the cylindrical portion (facing the target) and, optionally a tapered portion at the other end of the cylindrical portion. A second body of magnetic material, having an opposite magnetisation direction to the first body of the magnetic material, is preferably annular in shape and arranged to surround the first body such that a central axis of the annular shape and a central axis of the cylindrical shapes coincide.

Preferably the one or more bodies of magnetic material forming the magnetic element 6 have the same magnetisation direction. This simplifies the construction of the device because different parts of the device corresponding to different bodies of magnetic material do not repel each other.

Preferably, the one or more bodies of magnetic material forming the magnetic element 6 are arranged to have substantially cylindrical symmetry. In other words, the magnetic material forming the magnetic element 6 may have a circular cross section (a cross a longitudinal axis of the magnetic element 6). The axis of symmetry preferably coincides with the axis along which the peak force is located.

The magnetic material forming the magnetic element 6 is preferably a permanently magnetic material, e.g. NdFeB. This type of magnetic material is suitably strong. The magnetic material may have a magnetization of from 1.0 T to 1.4 T.

The magnetic element 6 may have a maximum width of from 2.5 cm to 15 cm (e.g. diameter in the case of an element having a circular cross-section). The magnetic element 6 may have a maximum length of from 2 cm to 10 cm. Such dimensions are suitable for holding the device in one hand. However, larger dimensions may be used for specific applications.

The configuration of the magnetic element 6 can be optimized by the routine described below.

A general expression for the magnetic force, F, on a single domain superparamagnetic particle with a moment of μ=M(B)V is given by

F=∇(μ·B)=V∇(M·B), (1)

where M is the magnetization of the particle, which depends on the field, V is the volume of the particle and B=μ₀H is the magnetic flux density, proportional to the applied field, H. As the particle is superparamagnetic, it is assumed that M and B are parallel. The magnetization of a superparamagnetic particle can be described using a Langevin function, L(y)=coth(y)−1/y,

$$M ( H ) = {M}_{s} L ( \frac{{M}_{s} V {μ}_{0} H}{{k}_{B} T} ) , \qquad ( 2 )$$

here M is the saturation magnetization of the particle, H is the applied field inside the particle and k_{B}T is the product of the Boltzmann constant and the temperature.

The field emitted by an array consisting of an arbitrary configuration of magnetic elements was calculated by breaking the magnet into a 3-dimensional arrangement of evenly distributed point moments, following a method described previously (Duck F. Physical Properties of Tissue: A Comprehensive Reference Book. Academic Press. 1990). Each moment emits a dipole field described by

$${B}_{i} ( {r}^{′} ) = \frac{{μ}_{0}}{4 π} ( \frac{3 {r}^{′} ( {μ}_{i} \cdot {r}^{′}}{{r}^{′5}} - \frac{{μ}_{i}}{{r}^{′3}} ) \qquad ( 3 )$$

where μᵢ=MdV is the point moment, M is the magnetization of the permanent magnet, dV is the volume occupied by the point and r′ is the position vector relative to the point moment. In the optimization routine, the normalized magnetic force due to the field emitted by an array of magnets on a superparamagnetic particle at a position of interest (POI) was calculated. The normalized magnetic force (or force per moment) is given by

$$\frac{F}{{M}_{s} V} = \frac{M}{{M}_{s}} ∇ ( B ) \qquad ( 4 )$$

and has units of Tm⁻¹. When the particle is saturated (M=Mₛ), the normalized force is equivalent to the field gradient emitted by the array. The magnetic particle considered here (e.g. superparamagnetic particle) has the same saturation magnetization as Fe₃O₄ at room temperature (Mₛ=4.7×10⁵ A m⁻¹) and a diameter of 10 nm.

The model was implemented using console applications written in the C# programming language (Microsoft Corporation, Redmond, Wash., USA).

The optimization routine is able to generate designs of arbitrarily-shaped magnet arrays to deliver the maximal normalized force on a particle at the POI (r_{POI}) given a series of design parameters, including the volume to be optimized, the nominal direction of normalized force (Fₙₒₘ), the volume of the magnet (V_{mag}), and the list of allowable magnetization directions contained within the array (FIG. 13). An initial array is constructed to occupy the volume to be optimized consisting of both magnetized and non-magnetized elements, with magnetized elements occupying the positions closest to the POI. The total volume of the magnetized elements is limited to V_{mag} at each step using a subroutine described below. The main routine then starts at the element closest to the POI and tests each allowable magnetization orientation, retaining the one that results in the best value of the optimized parameter, F(r_{POI})·Fₙₒₘ/MₛV generated by the whole array at the POI. The process is then repeated for the next closest element until all elements in the array have been treated. At this point, convergence is tested by comparing the attained array to the configuration of the starting array. If the routine has changed the array and resulted in an improvement in the optimized parameter, the process is rerun using the attained array as the new starting array and again starting from the element closest to the POI until all elements have been treated. If the routine does not change the array after treating all elements and the optimized parameter cannot be improved, the array is considered optimized.

Whenever the combined volume of all elements with a non-zero magnetization exceeds the V_{mag} parameter, a subroutine is performed in order to find and demagnetize the element that makes the least contribution to the normalized force. As the force depends on the gradient of the total field generated by the array at the POI, it cannot be assumed that this element is the element furthest from the POI. To find the element to demagnetize, each magnetized element is temporarily replaced by a non-magnetized element of the same volume and F(r_{POI})·Fₙₒₘ/MₛV for the remaining array is recorded. The element that makes the least difference to the optimized parameter when replaced by a non-magnetic element is demagnetized.

An example magnet output from the optimization routine is shown in FIG. 14.

The targeted delivery and retention of magnetic microbubbles 3 in a blood vessel flow phantom and in a model of liver perfusion ex vivo has been demonstrated (Stride E. et al, Magnetic targeting of microbubbles against physiologically relevant flow conditions, 2015).

The targeted delivery and retention of magnetic microbubbles in a PSA phantom system has also been demonstrated. A phantom model of PSA was devise with pulsatile flow condition, where direct injection of magnetic microbubbles can be achieved through a needle (FIG. 10A). Under a clinical relevant flow rate, there is retention of magnetic microbubbles in the target region (phantom pseudo aneurysm) at 100 seconds after magnetic microbubble injection (FIG. 10B). In contrast, there is no retention of normal microbubbles at 100 seconds after injection (FIG. 10C).

It has been shown that under pulsatile flow conditions, the retention of magnetic microbubbles within the target region under a magnetic field is persistent beyond 100 seconds (FIG. 10D, top line), whereas without a magnetic field, there is a gradual loss of magnetic microbubbles from the target region, with complete loss of magnetic microbubbles beyond 100 seconds (FIG. 10D, bottom line). Further details of the experiments performed are provided below.

Once administered, the microbubbles 3 may be ruptured using ultrasound to release the blood clotting agent and improve efficacy of the treatment.

The imaging ultrasound and the microbubble rupturing ultrasound may be provided by the same ultrasound element 7 or different ultrasound elements. The ultrasound element 7 and a magnetic element 6 may be provided within a single device, preferably a handheld device.

The location of the peak magnetic force along the axis and a focal point of the acoustic field may be substantially coincident. The magnetic element 6 and the ultrasound element 7 may be configured such that both the location of the peak magnetic force along the axis and a focal point of the acoustic field are located with the target volume of the pseudo aneurysm 1. This feature may be advantageous because such an arrangement ensures that the microbubbles 3 are subject to the maximum acoustic excitation and maximum magnetic force at the same location. This may improve the efficiency of the method. The focal point of the acoustic field may be the focal point when the ultrasound is applied to tissue, in water or in air, for example.

The pseudo aneurysm 1 may be located between 1 mm and 150 mm from the skin surface of a patient. Typically, a pseudo aneurysm 1 is between 5 mm and 30 mm from the skin surface. Accordingly, the location of the peak magnetic force and/or focal point of the acoustic field may be configured to be 1 mm and 50 mm from the surface of the device (and preferably between 5 mm and 30 mm).

The acoustic field and the magnetic field may be co-aligned. This may be advantageous because such an arrangement maximises the effectiveness of both the acoustic and magnetic fields at the target volume. This may also allow the size of the device to be minimised. For example, the magnetic element 6 and the ultrasound element 7 may be configured such that the axis along which the peak magnetic force is located and an axis through the focal point of the acoustic field and the ultrasound element 7 may be substantially co-aligned. For a substantially cylindrically symmetric magnetic element 6 and ultrasound element 7 the co-aligned axes may be axes passing through the centre of the magnetic element 6 and ultrasound element 7 respectively.

The ultrasound element 7 may comprise a piezo-electric transducer. This may be advantageous because this allows the ultrasound element 7 to be relatively compact in size. The ultrasound element 7 may generate ultrasound with a frequency of from 0.5 MHz to 15 MHz. The ultrasound element 7 may have a width of from 10 mm to 100 mm (e.g. diameter for an element having a circular cross-section).

The ultrasound element 7 may comprise a lens. The lens may focus the ultrasound towards the target. The lens may be formed from glass, for example. The lens may be concave. For example, the lens may have a flat surface in contact with an ultrasound source, such as a piezo electric transducer, and an opposing concave surface facing away from the ultrasound source. Such an arrangement may be advantageous because it allows the acoustic field generated by the ultrasound element 7 to be focused on the pseudo aneurysm thus maximising the effectiveness of the acoustic field in the treatment.

The microbubbles 3 used in the invention have a gas core. Generally, the gas for the gas core has a reflectivity that is suitable for the microbubble 3 to be effective as an ultrasound contrast agent. The gas is also selected for the application in which the microbubbles 3 are to be used. Typically, the gas is inert and biocompatible. For in vivo applications, the gas should not be toxic (in the amounts used) to the human or animal body. For cell transfection applications, the gas should not be cytotoxic. Suitable gases include, for example, air, nitrogen, carbon dioxide, oxygen, noble gases (e.g. helium, neon, argon, xenon), perfluorocarbon gases (e.g. perfluoropropane) and mixtures thereof. Preferably, the gas core of the microbubble 3 is selected from air, a noble gas, carbon dioxide, nitrogen, oxygen and mixtures thereof. More preferably, the gas core is selected from air, nitrogen, oxygen, and mixtures thereof. Most preferably the gas core is air.

The microbubbles 3 comprise a shell having a liquid layer. Typically, the microbubbles 3 further comprise an external coating. As described herein, a magnetic material 4 such as magnetic nanoparticles are typically suspended in the liquid layer of the shell. The liquid layer is selected to be chemically compatible with the coating material and should be suitable for the application in which the microbubbles are to be used (e.g. a non-toxic liquid is used for in vivo applications).

The liquid shell of the microbubble preferably comprises a hydrocarbon oil, preferably a non-volatile hydrocarbon oil, or derivatives thereof in a liquid layer. Suitable hydrocarbon oils include non-polar hydrocarbon oils (e.g. mineral oils) and hydrocarbon oils of plant or animal origin. Examples of non-polar hydrocarbon oils include isoparaffin, squalene, perhydrosqualene, paraffin oils, petroleum oils, hydrogenated or partially hydrogenated polyisobutene, isoeicosane, decene/butene copolymers, polybutene/polyisobutene copolymers and mixtures thereof. Examples of hydrocarbon oils of plant origin include wheatgerm oil, sunflower oil, grapeseed oil, groundnut oil, sesame seed oil, maize oil, apricot kernel oil, castor oil, shea oil, avocado oil, coconut oil, corn oil, olive oil, soybean oil, sweet almond oil, palm oil, rapeseed oil, cotton seed oil, hazelnut oil, macadamia oil, jojoba oil, alfalfa oil, poppy oil, evening primrose oil and mixtures thereof. It is preferred that the liquid shell comprises isoparaffin or soybean oil, more preferably a liquid layer of the shell is isoparaffin.

The shell may also comprise an external coating. Preferably, the external coating is a polymer, a surfactant or a lipid. Typically, the coating is an amphiphilic molecule, such as a medium or long chain aliphatic acid residue, a medium or long chain alkyl group and a hydrophilic group, or a polymer. Examples of suitable amphiphilic molecules are lipid or surfactants. Suitable lipids include phospholipids and/or glycolipids. Examples of lipids include phosphatidylcholine, phosphatidylethanolamine, phosphatidylserine, phosphatidylglycerol, phosphatide acid, phosphatidylinositol, dipalmitoylphosphatidylcholine (DPPC), distearoylphosphatidylcholine (DSPC), dimyristoyiphosphatidylcholine (DMPC), dioleylphosphatidylcholine (DOPE), dimyristoylphosphatidylethanolamine, dipalmitolphosphatidylethanolamine, distearoylphosphatidylethanolamine, lysolipids, fatty acids, cardiolipin, sphingomyelin, glycosphingolipids, glucolipids, glycolipids, sulphatides, lipids with ether and ester-linked fatty acids, polymerizable lipids, and combinations thereof. The lipids used may be of either natural or synthetic origin. There are also usable phospholipids derived from plants and animals such as egg yolk or soybeans and their hydrogenation products or hydroxide derivatives, so-called semi-synthetic phospholipids. Fatty acids constituting a phospholipid are not specifically limited, and saturated and unsaturated fatty acids are usable. Preferably, the material is hydrogenated L-α-phosphatidylcholine.

In some instances, the surface of the microbubble 3 may be modified with a polymer, such as, for example, with polyethylene glycol (PEG), using procedures readily apparent to those skilled in the art.

The magnetic material 4 may be composed of a variety of magnetic metals, such as iron, cobalt or nickel. Preferably, the magnetic material 4 comprises or consists of nanoparticles, preferably still ferromagnetic nanoparticles, such as iron oxide nanoparticles. The nanoparticles may contain various metal elements, such as Zn, Co and Ni, to control their magnetic characteristics. The iron oxide nanoparticles may comprise, as a main component, magnetite (Fe₃O₄), maghemite (γ-Fe₂O₃), Fe₃O₄ and/or mixed ferrite. The iron oxide typically comprises Fe₂O₃ or Fe₃O₄. The metal compound may comprise a mixed oxide of iron and another metal such as for instance a mixed oxide of (a) iron and (b) a second metal selected from cobalt, nickel, manganese, beryllium, magnesium, calcium, barium, strontium, copper, zinc, platinum, aluminium, chromium, bismuth, and a rare earth metal.

The magnetic material 4 may comprise or consist of “Janus” magnetic particles, i.e. particles whose surfaces have two or more distinct physical properties. For example, part of each particle may be hydrophobic and part hydrophilic. The hydrophobic part will encourage adsorption of the magnetic particle onto the microbubble. The blood clotting agents may be coupled to the hydrophilic part of the magnetic particle or the hydrophobic part. In this example, the magnetic particle fulfils two roles—coupling of the blood clotting agents and adsorption onto the microbubble 3 surface.

When the magnetic material 4 comprises or consists of Janus particles, the Janus particles can be prepared via any suitable process. Many routes to making Janus particles are known to those skilled in the art. For example, Janus particles can be produced via self assembly, by masking, or via phase separation. Janus particles can be prepared via self-assembly of substrates onto a nanoparticle such as a magnetic nanoparticle, wherein the substrates phase-separate when mixed such that the separation is maintained on assembly of the substrates onto the nanoparticle thus producing a Janus particle. Examples of Janus particles prepared by self-assembly include nanoparticles coated with immiscible block-copolymers or other ligands which typically show competitive adsorption on the nanoparticle surface. Janus particles prepared by masking can be obtained by trapping nanoparticles at the interface between two phases such as an immiscible liquid-liquid interface or a liquid-gas interface. Modification to part of the particle which is exposed to one side of the interface is then possible. Any suitable route can be used.

By way of non-limiting example, magnetic Janus nanoparticles can prepared by functionalising magnetic nanoparticles with a polymer such as poly-acrylic acid and then adsorbing the functionalised nanoparticles by electrostatic interaction onto beads such as silica beads. Chemical modification of the exposed polymer (e.g. via amidation) and subsequent dissociation from the beads leads to a Janus particle. Alternatively, magnetic Janus nanoparticles can prepared by stabilizing an oil-in-water emulsion with magnetic nanoparticles and then nucleating further nanoparticles (e.g. silver nanoparticles) in either the oil or preferably the aqueous phase such that selective reduction on the exposed surface of the magnetic nanoparticle leads to a Janus nanoparticle. In yet another alternative, magnetic Janus nanoparticles can be obtained by conjugating a first magnetic nanoparticle to a second nanoparticle obtained by phase reorganisation on the surface of the nanoparticle. For example, an Fe₂O₃ nanoparticle can be coated with e.g. S or Se and treated with a cadmium precursor such as Cd(acac)₂ such that on high-temperature reorganisation a magnetic Janus heterodimer of Fe₂O₃—CdS or Fe₂O₃—CdSe is formed.

Alternatively, magnetic heterodimeric Janus particles can be prepared by nucleation and subsequent epitaxial growth of a second magnetic nanoparticle on the surface of a first nanoparticle. For example, Au—Fe₃O₄ heterodimeric Janus particles can be prepared by nucleation and subsequent epitaxial growth of Fe₃O₄ on the surface of a gold nanoparticle, or via decomposition of Fe(CO)₅ on the surface of an Fe₃O₄ nanoparticle. Other magnetic Janus nanoparticles are commercially available.

For the avoidance of doubt, the term “nanoparticle”, as used herein, means a microscopic particle whose size is typically measured in nanometres. A nanoparticle typically has a particle size of from 0.5 nm to 1000 nm. A nanoparticle often has a particle size of from 1 nm to 200 nm, more typically from 1 nm to 100 nm. In the present invention, the average particle size of the magnetic nanoparticles is usually from 5 to 30 nm, preferably from 6 to 25 nm, more preferably from 8 to 12 nm. A nanoparticle may be spherical or non-spherical. Non-spherical nanoparticles may for instance be plate-shaped, needle-shaped or tubular. The term “particle size” as used herein means the diameter of the particle if the particle is spherical, or, if the particle is non-spherical, the volume-based particle size. The volume-based particle size is the diameter of a sphere that has the same volume as the non-spherical particle in question. Particle size can be determined using any suitable means such as dynamic light scattering or laser diffraction analysis using methods and equipment well known in the art.

The nanoparticles used in the invention can be made by any suitable method known in the art as may be identified by the skilled person. For example, nanoparticles may be made by attrition, where macro- or micro-scale particles are ground in a mill, such as a planetary ball mill. Nanoparticles may also be made by pyrolysis, wherein a vaporous precursor is forced through an orifice at high pressure and burned, with the resulting solids comprising oxide particles. Alternatively, a thermal plasma can be used to vaporize micrometer-size particles, or an radio frequency (RF) induction plasma torch can be used. In some aspects, inert-gas condensation can be used to make nanoparticles from metals with low melting points. Nanoparticles can alternatively be formed using radiation chemistry. In a preferred method, nanoparticles are derived from metal salts in solution, typically under anaerobic conditions. For example, iron oxide nanoparticles may be derived by co-precipitation from iron chloride hydrates in aqueous solution. Many such nanoparticles, including iron oxide nanoparticles, are also commercially available.

In order to aid suspension or dissolution of the magnetic nanoparticles in the shell, the magnetic nanoparticles may be coated. Typically a biocompatible coating is used. Substantially all of the nanoparticles are usually at least partially covered with the biocompatible coating. For example, at least 10% of the surface of each nanoparticle may be covered with the biocompatible coating. Often, at least 30% of the surface of each nanoparticle is covered with the biocompatible coating. For instance, at least 50% of the surface of each nanoparticle may be covered, or for example at least 75% of the surface such as at least 90% of the surface may be covered. For instance, each nanoparticle may be completely covered by the biocompatible coating.

The coating is selected to aid dispersibility or solubility of the magnetic nanoparticles and the coating used will depend on the liquid used to form part or all of the shell. The biocompatible coating may comprise materials such as carbohydrates, sugars (including long-chain sugars and the like), sugar alcohols, poly(ethylene glycols (PEGs), nucleic acids, amino acids, peptides, lipids and the like. Preferably, the magnetic nanoparticles are coated with one or more surfactants. Surfactants include oleic acid or salts thereof such as sodium oleate, dodecylamine, and sodium carboxy-methylcellulose, sodium cholesteryl sulfate and the like, and lipids. Suitable lipids include phospholipids and/or glycolipids. Examples of lipids include phosphatidylcholines (e.g. palmitoyloleoylphosphatidylcholine, palmitoyllinoleoylphosphatidylcholine, stearoyllinoleoylphosphatidylcholine, stearoyloleoylphosphatidylcholine, stearoylarachidoylphosphatidylcholine, and dipalmitoylphosphatidylcholine, hydrogenated soy phosphatidylcholine (HSPC)), phosphatidylethanolamine, phosphatidylserine, phosphatidylglycerol, phosphatide acid, phosphatidylinositol, dipalmitoylphosphatidylcholine (DPPC), distearoylphosphatidylcholine (DSPC), dimyristoyiphosphatidylcholine (DMPC), dioleylphosphatidylcholine (DOPE), dimyristoylphosphatidylethanolamine, dipalmitolphosphatidylethanolamine, distearoylphosphatidylethanolamine, lysolipids, fatty acids, cardiolipin, sphingomyelin, glycosphingolipids, glucolipids, glycolipids, sulphatides, lipids with ether and ester-linked fatty acids, polymerizable lipids, and combinations thereof. The lipids used may be of either natural or synthetic origin. There are also usable phospholipids derived from plants and animals such as egg yolk or soybeans and their hydrogenation products or hydroxide derivatives, so-called semi-synthetic phospholipids. Fatty acids constituting a phospholipid are not specifically limited, and saturated and unsaturated fatty acids are usable. The magnetic particles are preferably coated in oleic acid or a lipid.

The blood clotting agent may one or more of blood clotting factors I through XIII, including salts, derivatives and prodrugs thereof. A pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as oxalic, citric, fumaric, maleic, malic, ascorbic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases such as alkyl amines, aralkyl amines and heterocyclic amines. Hydrochloride salts and acetate salts are preferred, in particular hydrochloride salts. A “prodrug” means a precursor or derivative form of a blood clotting agent that has improved properties such as increased bioavailability, decreased toxicity, improved selectivity and the like as compared to the blood clotting agent and is capable of being enzymatically activated or converted into the more active form. Blood clotting factors I through XIII include thrombin: I—Fibrinogen; II—Prothrombin; III—Tissue factor; IV—Calcium ions (Ca²⁺); V/VI—Proaccelerin; VII—Proconvertin; VIII—Antihaemophylic factor; IX—Plasma thromboplastic component; X—Stuart factor; XI—Plasma thromboplastin antecedent; XII—Hageman factor; XIII—Fibrin stabilizing factor. The blood clotting factor may be recombinant or may be obtained from plasma such as from bovine or human plasma. Other blood clotting agents include desmopressin, vasopressin, vitamin K, collagen, oxidized cellulose, gelatin, chitosan, alginic acid, astringents such as tannic acid and vasoconstrictors such as epinephrine and angiotensin II, salts thereof, derivatives thereof and prodrugs thereof, and combinations of the above. Most preferably, the blood clotting agent is selected from thrombin, fibrin, and human blood coagulation factors such as human factor VII, including pharmaceutically acceptable salts, derivatives or prodrugs thereof. More preferably, the blood clotting agent is selected from thrombin and fibrin including pharmaceutically acceptable salts, derivatives or prodrugs thereof. Still more preferably, the blood clotting agent is thrombin or a pharmaceutically acceptable salt, derivative or prodrug thereof. Most preferably, the blood clotting agent is thrombin. Blood clotting agents suitable for use in the invention can be obtained using methods well known in the art, or are commercially available.

The magnetic material 4 may be attached to the blood clotting agent. The magnetic material may be attached to the blood clotting agent either directly or indirectly, e.g, via the microbubble. Therefore, the blood clotting agent may be attached to the microbubble but not directly conjugated to the magnetic material. Alternatively the blood clotting agent may be directly conjugated to the magnetic material. As noted above, the magnetic material is typically suspended in the liquid layer of the shell of the microbubble 3.

The blood clotting agent may be attached to the magnetic material 4 via any suitable means. For example, the blood clotting agent may be covalently or non-covalently bonded to the magnetic material. Alternatively, the blood clotting agent may be bonded via a linker group or a spacer group to the magnetic material. The blood clotting agent may be bonded to the magnetic material 4, e.g. magnetic nanoparticles, via disulphide bond coupling or electrostatic coupling. Alternatively, the magnetic particles 4 may be coated in an albumin (or other protein) in which case the blood clotting agent can bind (e.g. by non-covalent binding) to the albumin or other protein used to coat the particles. The blood clotting agent may be bound to the microbubble 3 via adsorption of the drug onto the surface of the coated magnetic particle. The adsorption may be provided by for example, avidin-biotin coupling, malemide coupling and/or electrostatic coupling. Alternatively, the blood clotting agents may be encapsulated in liposomes and/or polymeric particles. The encapsulated blood clotting agents may be coupled to the microbubble 3 surface via avidin-biotin coupling, malemide coupling and/or electrostatic coupling. In other embodiments, the blood clotting agent is attached to the coating of the microbubble 3. In another embodiment, the blood clotting agents may be suspended or dissolved in a liquid layer of the microbubble shell. The blood clotting agents should be chemically compatible with the liquid layer. It may be necessary to modify the blood clotting agents to aid its suspendability or solubility in the liquid layer using standard methods known in the art.

Loading of microbubbles with the blood clotting agent can be achieved in multiple ways, as illustrated in FIG. 8. The skilled person will readily appreciate that any suitable coupling method between the microbubble and blood clotting agent can be used. Exemplary loading methods are provided below, however any loading method which does not alter the therapeutic efficacy of the blood clotting agent may be used.

1) Direct Incorporation:

Blood clotting agents can be directly incorporated into the shell of the microbubble. Lipid and protein microbubbles have an amphiphilic structure, which allows loading of amphiphilic, hydrophilic, or hydrophobic drugs. Albumin in particular has excellent inherent capability as a drug carrier. This method is simple to employ and drugs are rapidly released upon ultrasonic activation. The drug loading capacity of microbubbles can be estimated using known parameters of drug loading area, hydrophobicity, and drug solubility; however, quantification of the loaded drug, by methods known in the art, is simpler and more accurate.

2) Electrostatic Incorporation:

Direct incorporation of hydrophilic blood clotting agents or carriers therefor onto the surface of microbubbles can also be achieved by electrostatic interaction. Use of a microbubble with the opposite charge of the drug or drug carrier—which may itself be modified to achieve the desired charge—creates an electrostatic force that binds the two components together. In microbubble chemistry, hydroxyl, phosphate, and carboxylic acid groups can be used to confer a negative charge. Positive charges can be provided by protonated nitrogen groups such as primary, secondary, tertiary or quaternary amines (e.g., NH₃, trimethyl amine, etc). The presence of these groups can be confirmed by many standard chemical tests; however, the charge conferred is of primary interest. Zeta potential measurements provide a useful means for determining the charge on a molecule. Measurement of zeta potentials is known in the art; for example, the zeta-potential of particles can for example be determined using laser Doppler electrophoresis (LDE), which measures MB electrophoretic mobility in an electric field by light scattering and calculating its zeta potential. In this method the buoyancy force acting on the bubbles can significantly affect their measurement as commercial systems typically have a vertical. An alternative approach that has been reported uses a simple microfluidic device for obtaining the zeta potential of commercially available microbubbles. The microbubbles are suspended in a horizontal microchannel under an electrical field and optically tracked as illustrated in FIG. 9. The velocity of the bubbles is then used to calculate their zeta potentials. By comparing LDE and the homemade device, a tenfold improvement in measurement variation was achieved. It should be noted that coating of the channel is needed to avoid microbubbles sticking to the wall as well as reducing electro-osmotic flow on the walls.

There are some challenges associated with measuring microbubbles in commercial machines. For example, due to their semi-rigid cross-linked nature, shell remnants of destroyed protein microbubbles should yield a relatively good estimation of zeta potential, but this is not necessarily the case with all materials. Consideration must also be given to both the buffering solution and effect in plasma, as the charge of a molecule will change with pH and ionic strength. For example, site-specific release in tumors can be influenced by the high metabolic rate in tumor cells and excretion of H⁺ ions resulting in acidification of the tumor environment.

3) Conjugation of Drug Carriers:

It is sometimes preferable to permanently attach the blood clotting agent to a microbubble shell. In these instances, the drug is typically loaded into a secondary drug carrier such as polymeric nanoparticles or liposomes, and attached to the microbubble via electrostatic or chemical conjugation. This improves colocalization of the drug with the microbubble at the site of activation and can help reduce systemic toxicity by drug encapsulation. In addition, conjugation of drug carriers to the shell can potentially increase the loading capacity per microbubble in comparison with incorporation into the shell. For example, up to 10000 liposomes of 100 nm have been reported as being loaded via avidin-biotin linkages on to single microbubbles with mean diameters of 1.7 μm. Compared with microbubbles only, the attachment of these liposomes can give a nearly 35-fold increase in the potential surface area alone. Many conjugation techniques are available and many protocols exist for their use, in both the literature and from commercial suppliers. Direct conjugation of a carrier to a shell is possible via functional ligands; however, linker molecules are typically employed to separate the drug carrier from the shell. A popular linker is polyethyleneglycol (PEG), due to its low toxicity, low cost, the wide availability of different forms and conjugates, and the ease of manipulation. PEG chains with two differing functional groups—a heterobifunctional ligand—can allow for very precise loading of desired carriers, with low interference. For protein microbubbles, the PEG linker should be conjugated onto the protein shell postproduction. Preproduction conjugation may interfere with functional groups required for stable shell synthesis, although, with controlled reactions, incomplete usage of functional groups could be attempted. For lipid microbubbles, conjugation can be performed pre-production to reduce the number of handling steps of fragile microbubbles. Alternatively, PEG-lipid conjugates with functional groups are already widely available and can be incorporated directly during microbubble production. Unreacted PEG molecules can be quantified by refractive index detection after separation from microbubbles by washing and size exclusion chromatography. In addition, quantification of the relative and absolute amounts of specific lipids in incorporated into the shell can be performed using techniques such as gas chromatography (GC), Fourier transform infrared (FTIR) spectroscopy, or high performance liquid chromatography (HPLC) on washed microbubbles. HPLC combined with an evaporative light scattering (ELS) detector has been used to determine the amounts of lipids in commercial and research microbubble formulations. Alternatively, the available functional groups on the microbubbles can be characterised.

4) Avidin-Biotin:

Avidin-biotin is one of the strongest noncovalent bonds commonly available to researchers. It is simple to use, and many PEG linkers or PEG-lipid conjugates with either biotin or avidin ligands are available. It is highly tolerant to a wide range of buffering conditions and is common in the preparation of novel microbubble delivery or imaging agents.

Qualitative determination of incorporation into a microbubble shell can be achieved by widely available fluorescent biotin or avidin. Quantitatively, colorimetric and fluorimetric tests using 2-(4′-hydroxyazobenzene) benzoic acid (HABA) are available to rapidly determine biotin levels in a sample. This has been performed for biotinylated albumin to determine the binding efficiency of the conjugation of biotin to albumin subsequently used to create microbubbles and on antibodies to be conjugated to microbubbles via avid in-biotin. However, considering the low proportion of biotinylated lipids used in formulations, typically ranging from 5% to 10% molar ratio, microbubbles have not been directly investigated in this manner yet as even fluorimetric HABA assays may struggle to determine biotin quantities.

Alternatives to avidin known in the art include strepatavidin and neutravidin.

5) Carbodiimide Chemistry:

In carbodiimide chemistry, a carboxylic acid group is reacted with 1-ethyl-3-(3-dimethylaminopropyl) carbodiimide (EDC), typically in the presence of N-hydroxysuccinimide (NHS) or N-hydroxy-sulfosuccinimide (sulfo-NHS) to improve yield, forming a ligand capable of forming a covalent bond with amine groups. Carbodiimide chemistry is popular as a protein linker ligand, as amine and carboxylic acid groups are common in protein residues. It is also popular for adding other functional groups to a molecule, e.g., sulfhydryls, avidin, biotin, etc. Typically, linkers use NHS due to its increased stability; however, the reaction can be performed on carboxylic acid or amine groups already present on the molecules without further manipulation. A lipid or protein microbubble shell with available carboxylic acid groups can be modified by EDC-NHS to bind amine functionalized linkers or drug carriers, or vice versa. Lipid-PEG-NHS conjugates are available for direct incorporation during lipid microbubble manufacture. EDC, and its hydrolysed decay products, can be detected by mass spectroscopy and colorimetric methods. NHS and sulfo-NHS have strong absorbance at 260 nm under basic conditions, and their unreacted conjugates can be isolated and detected by fluorescence assay or HPLC-UV using a reversed phase column. Alternatively, during a reaction, released NHS and sulfo-NHS can be quantified by HPLC-UV using a hydrophilic interaction chromatography column. Indirectly, a before and after assay of available amine, e.g., 2, 4, 6-trinitrobenzene sulfonic acid (TNBS) assay, and carboxylic acid groups, e.g., 5-bromomethylfluorescein assay, can determine site usage. Finally, an amine displaying a fluorescent compound can be targeted to the activated EDC/NHS groups to determine availability on the shell.

6) Sulfhydryl Linkages:

On proteins, sulfhydryl groups are less common than amine groups, increasing reaction selectivity and potentially reducing interference of protein microbubble formation. There are also many methods for adding sulfhydryls to target drug carriers via other functional groups, for example, Traut's reagent or N-succinimidyl S-acetylthioacetate variants that target amine groups. The availability of these sulfhydryl groups can be quantified by several methods, and commercial assay kits are available. Several functional groups are capable of binding to sulfhydryl groups and the use of maleimide and pyridyl disulphide groups have been reported for microbubbles. Maleimide reagents are more stable than EDC/NHS and form permanent thioether linkages. The presence of maleimide groups can be detected using commercially available fluorimetric assay kits or spectrophotometric approaches. The maleimide group remains in the compound post reaction as a small linker.

Pyridyl disulphide forms a direct disulphide bond with sulfhydryl groups with no linker length and release of the pyridine-2-thione, which can be monitored by absorbance at 340 nm. This release can also be used to quantify the availability of functional groups as the pyridyl disulphide bond cleaves under reducing conditions, for example, in the presence of dithiothreitol, glutathione, or tris(2-carboxyethyl)phosphine hydrochloride. Although pyridine-2-thionehas a relatively low molar extinction coefficient that may hinder monitoring of the reaction at low concentrations, UV/VIS detection at 270 and 340 nm can be used to monitor the conjugation of pyridyl disulphide microbubbles to thiolated liposomes. The bond formed with the drug carrier is similarly not stable, which may allow drug release for example in the reducing environment of tumors.

7) Incorporation in an Oil Layer:

The blood clotting agent can be loaded into an oil or other liquid layer trapped beneath the shell of the microbubble (i.e. on the interior of the microbubble). Any biocompatible oil can be used. Suitable oils are discussed above.

Preferably, therefore, in one embodiment, the magnetic material is an oleic acid or lipid coated nanoparticle, and the blood clotting agent is coupled to the microbubble and/or the nanoparticle via a disulphide bond or a maleimide linker, preferably a disulphide bond. Preferably, in this embodiment, the nanoparticle is an iron oxide nanoparticle or a janus nanoparticle as described herein, preferably an iron oxide nanoparticle. Preferably, in this embodiment, the blood clotting agent is thrombin or a pharmaceutically acceptable salt, derivative or prodrug thereof, preferably thrombin.

In another embodiment, the magnetic material is a nanoparticle coated with albumin or another protein and the blood clotting agent is attached to the nanoparticle by physical adsorption.

Preferably, in this embodiment, the nanoparticle is an iron oxide nanoparticle or a janus nanoparticle as described herein, preferably an iron oxide nanoparticle. Preferably, in this embodiment, the blood clotting agent is thrombin or a pharmaceutically acceptable salt, derivative or prodrug thereof, preferably thrombin.

In another embodiment, the nanoparticle is coated in avidin, streptavidin or neutravidin and the blood clotting agent is conjugated to biotin such that the blood clotting agent is attached to the nanoparticle by interaction of the avidin/streptavidin/neutravidin and the biotin. Preferably, in this embodiment, the nanoparticle is an iron oxide nanoparticle or a janus nanoparticle as described herein, preferably an iron oxide nanoparticle. Preferably, in this embodiment, the blood clotting agent is thrombin or a pharmaceutically acceptable salt, derivative or prodrug thereof, preferably thrombin.

In still another embodiment the magnetic material comprises nanoparticles suspended in the liquid layer of the shell of the microbubble and the blood clotting agent is encapsulated in a liposome or is attached to or encapsulated in a polymeric particle, such that the liposome or polymeric particle can be coupled to the bubble surface either electrostatically or chemically e.g. via the coupling methods described herein. Preferably, in this embodiment, the nanoparticle is an iron oxide nanoparticle or a janus nanoparticle as described herein, preferably an iron oxide nanoparticle. Preferably, in this embodiment, the blood clotting agent is thrombin or a pharmaceutically acceptable salt, derivative or prodrug thereof, preferably thrombin.

The properties and parameters of the microbubbles 3 may be optimised in consideration of their use with a magnetic field.

In order to determine the parameters of a microbubble structure or design, which is optimised for magnetic actuation in a liquid and is to be ruptured by ultrasound, then the equations describing the microbubble need to be considered. Some of these equations may constrain the design of the microbubble and are considered below.

### 1. Microbubble Buoyancy

The weight of an individual microbubble, W, is given by

W=−4/3πg[ρ_{g}R₁³+(R₂³−R₁³)((1−α)ρ₀+αρₙₚ)]

and the buoyant force F_{BW} of the microbubble in a liquid, such as water, is given by

F_{BW}=4/3ρgρ₁R₂³

where g is acceleration due to gravity, μg, ρ0, ρₙₚ and ρᵢ are the densities of the filling gas (gas core of the microbubble), the nanoparticle suspending liquid of the microbubble shell, the nanoparticle material and the carrier liquid (i.e. the liquid in which the microbubble is to be suspended in use) respectively and a is the volume fraction of nanoparticles in the shell. The outer layer of the shell that surrounds the solvent layer, such as the phospholipid coating shown in FIG. 3, has a relatively negligible thickness (˜1.5 nm) and its contribution to the weight of the microbubble may be neglected.

### 2. Magnetic Force

The force on an individual magnetic microbubble on application of a magnetic field may be represented by:

$${F}_{M} = - \frac{4 π χ ( B \cdot ∇ ) B α ( {R}_{2}^{3} - {R}_{1}^{3} )}{3 {μ}_{0}}$$

where B is the magnetic flux or field strength at the location of the microbubble, χ is the effective volumetric susceptibility of the magnetic nanoparticles suspended in the solvent layer, and μₒ is the permeability of free space.

### 3. Acoustic Excitation and Scattering

When a microbubble is exposed to ultrasound, it undergoes volumetric oscillations. The enhancement in cell membrane permeability that is observed for non-magnetic microbubbles is thought to be related to the volumetric oscillations of the microbubbles. The amplitude of these oscillations must therefore be sufficient to promote transfection enhancement.

The equation of motion describing microbubble oscillations is non-linear (Stride, E; Phil. Trans. Roy. Soc. A., 2008, 366, 2103-2115), but a linearised form can be written as:

m\ddot{x}+b\dot{x}+kx=p(t)

where p(t)=p_{jnc} sin(ωt) and p_{inc} is the pressure amplitude of the applied ultrasound field and ω is its frequency in radians/s. The instantaneous radius of the bubble R is given by R=R₀₁x(t), where R₀₁ is the initial value of the radius of the gas core and x(t) represents a small change in R. The coefficients m, b and k are given by:

$$m = {ρ}_{s} {R}_{01}^{2} ( 1 + \frac{{R}_{01} ( {ρ}_{s} - {ρ}_{l} )}{{R}_{02} {ρ}_{s}} )$$

$$b = \frac{4}{{R}_{02}^{3}} ( {μ}_{l} {R}_{01}^{3} + {μ}_{s} \frac{4}{3} π ( {R}_{02}^{3} - {R}_{01}^{3} ) + \frac{{R}_{01}^{3}}{{R}_{02}} {η}_{s 0} )$$

$$k = 3 κ ( {p}_{0} + \frac{2 {σ}_{01}}{{R}_{01}} + \frac{2 {σ}_{02}}{{R}_{02}} ) + \frac{4 {KT}_{0}^{y + 1} {R}_{01}^{3}}{{R}_{02}^{4} ( y + 1 )} - \frac{2 {σ}_{01}}{{R}_{01}} + \frac{2 {σ}_{02} {R}_{01}^{3}}{{R}_{02}^{4}}$$

where ρₛ=((1−α)ρ₀+αρₙₚ) is the effective density of the liquid layer around the bubble, _{μs} is its effective viscosity, _{μι} is the viscosity of the liquid in which the bubble is suspended and ηₛₒ is the viscosity of the outer coating layer, such as a surfactant, K is the polytropic constant for the gas core, p₀ is the ambient pressure, σ₀₁ and σ₀₂ are the initial interfacial tensions at the inner and outer surfaces of the liquid layer. FQ is the initial concentration of the material that forms the outer coating layer (e.g. surfactant on the bubble surface), y and K are constants for the material that forms the outer coating layer and R₀₂ is the initial radius of the microbubble.

Solving equation of motion in linearised form above, the amplitude of radial oscillation of the bubble, x, can be estimated as

$$X = \frac{{p}_{inc}}{\sqrt{( {( k - m {ω}^{2} )}^{2} + {b}^{2} {ω}^{2} )}}$$

Similarly, the scattering cross-section which determines whether or not microbubbles can be detected from the scattered ultrasound field that they produce can be written as

$${σ}_{scat} = \frac{4 π {ω}^{4} {X}^{2} {ρ}_{w}^{2} {R}_{01}^{6}}{{p}_{inc}^{2}} .$$

### 4. Viscous Drag

The viscous drag force on a microbubble suspended in a liquid having a net flow of velocity u is given by

$${F}_{D} = \frac{{\dot{u}}^{2} {C}_{D} π {R}_{{ }^{02}}^{2}}{2}$$

and for a low velocity (laminar) flow

F_{D}=6πμ₁uR₀₂

where C_{D} is the drag coefficient (approximation for a solid sphere) and μₗ, is the viscosity of the liquid.

Parameters for Optimum Microbubble Designs

Whilst it is desirable to increase the magnitude of the magnetic force on an individual microbubble by increasing its magnetic nanoparticle content, there will be a maximum volume fraction of nanoparticles that can be suspended in the shell before precipitation of the nanoparticles occurs. Moreover, the volume fraction cannot increase indefinitely because the viscosity of the liquid in which the nanoparticles are suspended will become so high that it will effectively be a solid. Thus, a first condition that influences the microbubble design is that the volume fraction a of magnetic nanoparticles in the solvent layer of the microbubble shell is

0<α<0.2. (i)

In order that the magnetic microbubbles are directable and actuatable in the environment of use, the microbubbles must contain sufficient magnetic material to enable them to be actuated by an applied magnetic field. Further, the force provided by this field must be sufficient to overcome microbubble weight and the microbubble buoyancy. A second condition that determines the microbubble design is that:

|F_{M}|>|F_{BW}+W|. (ii)

Microbubbles having marginal buoyancy are more responsive to the applied magnetic field because they neither sink nor rise rapidly. A third condition that influences the design of the microbubble is that:

$$- 1 < \frac{{F}_{BW} + W}{W} < 0 \qquad ( iii )$$

as above.

If the microbubble is initially introduced away from the target site, then ideally it should be moveable toward the target site using a magnetic field. As the bubble moves through the liquid in which it is suspended, it will experience drag resistance from the viscosity of the liquid and the flow of that liquid. A fourth condition is that magnetic actuation of the microbubble should be sufficient to overcome the drag force of the microbubble in the carrier liquid, such that:

|F_{M}|>|F_{D}|. (iv)

It is preferable that the microbubble is able to be monitored using ultrasound. The microbubble must, however, be capable of being ruptured when exposed to high intensity ultrasound at a practicable frequency that can be generated by available apparatus. As explained above, the uptake of a therapeutic agent by cells is improved by sonoporation. The microbubble must therefore contain a sufficient volume of gas in order that it can undergo volumetric oscillations of sufficient amplitude in response to an ultrasound field. A fifth condition is therefore that the microbubble scattering cross section satisfies the condition:

$${σ}_{scat} \geq \frac{4 π {ω}^{4} {ρ}_{l}^{2} {R}_{01}^{6}}{( {( k - m {ω}^{2} )}^{2} + {b}^{2} {ω}^{2} )} ; \\ where \\ m = {ρ}_{l} {R}_{{ }^{01}}^{2} , b = 4 {μ}_{ι} + \frac{3.6 \times {10}^{- s}}{{R}_{01}} and k = 3 \times 1 {0}^{5} + \frac{0.24}{{R}_{01}} . \qquad ( v )$$

(ω is the frequency of the ultrasound, ρ₁ is the density of the carrier liquid, R₀₁ is the initial radius of the gas core of the microbubble and μι is the viscosity of the carrier liquid (the liquid in which the microbubble is suspended)).

If the microbubbles are to be administered intravenously, then the microbubbles must not be too large, otherwise the amount of gas introduced into the blood stream might be harmful and cause an embolism. Thus, there is a maximum diameter of the microbubbles for in vivo applications, which, generally in practice, should not exceed 8 μm.

Determination of Optimum Microbubble Design Parameters

By applying each of the above conditions, a range of microbubble designs can be determined that are optimised for delivery of a therapeutic agent or for cell transfection applications, when used with a magnetic field of known strength, ultrasound of a known frequency and the flow velocity in the carrier liquid is known. A range of optimised microbubble designs may be determined from the parameters ψ, φ and λ, which are defined below. ψ is defined as:

$$ψ = \frac{{F}_{BW} + W}{W} = 1 - \frac{{ρ}_{l} {R}_{{ }^{2}}^{3}}{( {ρ}_{g} {R}_{1}^{3} + ( {R}_{2}^{3} - {R}_{1}^{3} ) ( ( 1 - α ) {ρ}_{0} + {αρ}_{np} ) )} .$$

For marginally buoyant microbubbles (i.e. when |W|<F_{BW}<|2W|) ψ is between 0 and −1. φ is defined as:

$$φ = \frac{{F}_{M}}{{F}_{BW} + W} = \frac{- χ ( B \cdot ∇ ) B α ( {R}_{2}^{3} - {R}_{1}^{3} )}{g {μ}_{0} \{ {ρ}_{l} {R}_{{ }^{2}}^{3} - ( \begin{matrix}{ρ}_{g} {R}_{1}^{3} + \\ ( {R}_{2}^{3} - {R}_{1}^{3} ) ( ( 1 - α ) {ρ}_{0} + {αρ}_{np} )\end{matrix} ) \}} .$$

For microbubbles to be actuateable by an applied magnetic field it is necessary that φ is less than −1.
λ is defined as:

$$λ = \frac{{F}_{M}}{\vert {F}_{D} \vert} = \frac{- 2 χ ( B \cdot ∇ ) B α ( {R}_{2}^{3} - {R}_{1}^{3} )}{9 {μ}_{l} {R}_{2} {μ}_{0} \vert u \vert} .$$

In order that the microbubbles are responsive and moveable in the carrier liquid when a magnetic field is applied, the magnetic force must be sufficient to overcome the drag viscosity of the microbubble in that liquid. Thus, the parameter λ should be less than than −1.

As an example, an optimised microbubble formulation was determined using the parameters set out in Table 1. A graph of shell thickness as a fraction of gas core radius against nanoparticle volume fraction was plotted for each of ψ, φ and λ, with ψ, φ and λ each taking various values, see FIGS. 4 to 6. The highest value shown on the x-axis of each graph is 0.2 to represent the condition that 0<α<0.2.

TABLE 1

| Parameter | Value | |
|---|---|---|
| ρₗ (liquid = water) | 1000 | kg/m³ |
| μₗ (liquid = water) | 0.001 | Pa s |
| ρ_{g} (gas = air) | 1.24 | kg/m³ |
| g | 9.81 | m/s² |
| p₀ | 10⁵ | Pa |
| ρ₀ (isoparaffin) | 700 | kg/m³ |
| ρₙₚ (iron oxide) | 5100 | kg/m³ |

| | |
|---|---|
| nartoparticle radius | 5.00 × 10⁻⁹ m |
| μ₀ | 1.26 × 10⁻⁶ Tm/A |
| X for B = 0.4 T | 0.089 |

In FIG. 4, there are lines for ψ=0 and ψ=−1. These lines represent the limits for microbubbles that satisfy the conditions for w set out above. The region between the lines for ψ=0 and ψ=−1 therefore represents the range of microbubble formulations that satisfy the −1<ψW<0.

Similarly, FIGS. 5 and 6 show the limits for the parameters φ and λ that satisfy the condition φ<−\ and λ<−\ for a given magnetic field and flow velocity of the liquid in which the microbubbles are suspended (in this case, (B. ∇) B=18 T²/m and a flow velocity of water of 10⁻⁴ m/s).

A formulation map (FIG. 7) may be obtained by combining on a single graph, the individual graphs for ψ, φ and λ, such as those shown in FIGS. 4 to 6. The formulation map defines a range of microbubble formulations that satisfy the conditions set out above for each of ψ, φ and λ. The inventors have surprisingly found that the requirement that the magnetic microbubbles are marginally buoyant (represented by the conditions for parameter ψ) constrains the design of a microbubble for the applications described herein.

It is desirable that the values of a and the shell thickness as a fraction of gas core radius (ξ) are as small as possible, whilst still satisfying the limits set out for ψ, φ and λ. For example, using the formulation map in FIG. 7, it can be seen that a microbubble having a volume fraction of magnetic nanoparticles of 0.1 and a gas core radius of 1 μm can have a shell thickness of approximately 0.23 μm for the magnetic field and flow velocity in water specified above.

### Therapeutic Efficacy

The compositions of the present invention are therapeutically useful. The present invention therefore provides compositions as described herein, for use in treating pseudo aneurysm in a subject in need thereof.

Also provided is a pharmaceutical dosage form comprising a composition of the invention together with a pharmaceutically acceptable carrier, diluent or excipient. Typically, the composition contains up to 85 wt % of the composition of the invention. More typically, it contains up to 50 wt % of the composition of the invention. Preferred pharmaceutical compositions are sterile and pyrogen free. Further, when the pharmaceutical compositions provided by the invention contain a blood clotting agent which is optically active, the compound of the invention is typically a substantially pure optical isomer.

The composition of the invention may be provided as a kit comprising instructions to enable the kit to be used in the methods described herein or details regarding which subjects the method may be used for.

As explained above, the composition of the invention are useful in treating pseudo aneurysm. Pseudo aneurysm is also referred to as false aneurysm. Diagnosis of pseudo aneurysm can be performed by techniques known in the art such as Duplex ultrasonography, CT angiogram or conventional angiogram techniques.

The compositions of the invention may be used as standalone therapeutic agents. Alternatively, they may be used in combination with other therapeutic agents or techniques. For example, the subject to be treated may also receive or have received or been considered for treatment using an endovascular stent; ultrasound probe compression of the neck of the pseudoaneurysm; or surgical ligation (with or without distal bypass). The subject may not have received treatment using an endovascular stent; ultrasound probe compression of the neck of the pseudoaneurysm; or surgical ligation (with or without distal bypass). The subject may not be clinically able to receive treatment using an endovascular stent; ultrasound probe compression of the neck of the pseudoaneurysm; or surgical ligation (with or without distal bypass).

The compositions of the invention are useful in treating pseudo aneurysm. The present invention therefore provides a composition of the invention for use in the treatment of pseudo aneurysm. The invention also provides the use of a composition of the invention in the manufacture of a medicament for treating pseudoaneurysm. The invention also provides a method of treating pseudoaneurysm, said method comprising administering a composition of the invention to a subject in need thereof.

In one aspect, the subject is a mammal, in particular a human. However, it may be non-human. Preferred non-human animals include, but are not limited to, primates, such as marmosets or monkeys, commercially farmed animals, such as horses, cows, sheep or pigs, and pets, such as dogs, cats, mice, rats, guinea pigs, ferrets, gerbils or hamsters. The subject can be any animal that is capable of being infected by a bacterium.

The compositions described herein are useful in the treatment of pseudo aneurysm which most commonly occurs due to femoral artery puncture during cardiac catheterisation. About 100,000 cardiac catheterisations are performed in England each year. Up to 2% of cardiac catheterisations lead to pseudoaneurysm formation. Pseudoaneurysms may also occur following other procedures that involve puncture of an artery, including removal of an arterial blood pressure line or intra-aortic balloon pump, or following accidental trauma. The compositions described herein can therefore be used in the treatment of pseudo aneurysm in a subject who has previously received treatment involving cardiac catherterisation. The compositions described herein can also be used in the treatment of pseudo aneurysm in a subject who has previously received treatment involving the puncture of an artery. The patient may have experienced trauma such as accidental trauma such as blunt trauma (e.g. to an extremity), or penetrating trauma (e.g. gunshot or blast injury).

The compositions of the invention are particularly beneficial in the treatment of pseudoaneurysm in subjects having an arteriovenous fistula (communication between an artery and vein), in addition to the pseudoaneurysm. This occurs with about 10% of pseudoaneurysms. Conventional treatment using thrombin via UGTI is typically unsuitable for such patients as the thrombin can then enter the venous circulation and possibly lead to distant thrombosis. The arteriovenous fistula is typically in the region of the pseudo aneurysm.

The compositions of the invention may be administered in a variety of dosage forms. However, the composition of the invention is most commonly administered parenterally, whether subcutaneously, intravenously, intramuscularly, intrasternally, transdermally or by infusion techniques. Most commonly the composition of the invention is administered via intravenous administration, such as via ultrasound-guided injection (e.g. using real-time Doppler ultrasound guidance).

The composition of the invention is typically formulated for administration with a pharmaceutically acceptable carrier or diluent. For example, suspensions and emulsions may contain as carrier, for example a natural gum, agar, sodium alginate, pectin, methylcellulose, carboxymethylcellulose, or polyvinyl alcohol. The suspension or solutions for intramuscular injections or inhalation may contain, together with the active compound, a pharmaceutically acceptable carrier, e.g. sterile water, olive oil, ethyl oleate, glycols, e.g. propylene glycol, and if desired, a suitable amount of lidocaine hydrochloride. Solutions for inhalation, injection or infusion may contain as carrier, for example, sterile water or preferably they may be in the form of sterile, aqueous, isotonic saline solutions. Pharmaceutical compositions suitable for delivery by needleless injection, for example, transdermally, may also be used.

A therapeutically or prophylactically effective amount of the composition of the invention is administered to a subject in need thereof. A therapeutically effective amount is an amount effective to ameliorate one or more symptoms of the disorder i.e. the pseudo aneurysm. The dose may be determined according to various parameters, especially according to the compound used; the age, weight and condition of the subject to be treated; the route of administration; and the required regimen. A physician will be able to determine the required route of administration and dosage for any particular subject. A typical dose of the composition of the invention is determined to correspond to a dosage of the blood clotting agent of from about 0.01 to 100 mg per kg, preferably from about 0.1 mg/kg to 50 mg/kg, e.g. from about 1 to 10 mg/kg of body weight, according to the activity of the specific agent, the age, weight and conditions of the subject to be treated, the type and severity of the disease and the frequency and route of administration. Often, a single administration is sufficient to treat the pseudoaneurysm. If subsequent administrations are required then dosages may be administered for example daily.

### Experimental Details

A suspension of microbubbles 3 may be prepared using standard methods known in the art, provided that the method takes into account the amount of magnetic nanoparticles and the amount of liquid for the shell needed to prepare a suspension of optimised microbubbles 3. A preferred way of preparing a suspension of the microbubbles 3 comprises the step of shaking and/or sonicating an aqueous solution comprising a material for coating the microbubble shell, the magnetic nanoparticles and the liquid for the shell, then, typically, allowing the solution to settle before extracting a lower part of the solution. The suspension may be prepared by forming, in an aqueous solution, an emulsion of the ingredients for forming the microbubble 3, followed by shaking and/or sonicating. The sonication and/or shaking step is typically carried out under an atmosphere of the gas that is to be trapped as the gas core of the microbubbles. Sonication or shaking may be performed until formation of a foam.

The gas for the gas core of the microbubble 3 suspension may be bubbled through the aqueous solution used to prepare the suspension in addition to, or as an alternative to, carrying out sonication and/or shaking under an atmosphere of the desired gas. Suitable gases for the gas core are described below. Generally, a material for coating the microbubble shell is added to the aqueous solution, such as phosphate buffered saline (PBS), used to prepare the suspension of microbubbles 3. The coating material forms the outer layer of the microbubble shell, such that the magnetic nanoparticles are suspended in a liquid layer around the gas core. The coating material may form an additional inner layer in the shell, which separates the core and the solvent layer. Examples of suitable coating materials are described below. The suspension or solution of the nanoparticles may be added to the aqueous solution for preparing the microbubbles 3 before or after the coating material is added. Examples of suitable magnetic nanoparticles are described below.

In order to form microbubbles 3, the shell liquid and shell coating material are selected to form a stable emulsion in an aqueous solution. Generally, the liquid used to form the liquid layer of the microbubble shell is a hydrophobic solvent. The liquid is selected for its chemical compatibility with the magnetic nanoparticles and examples of suitable liquids are described below.

A suspension of microbubbles 3, which may have been prepared according to the sonication/shaking method outlined above, or according to a method known in the art, may be filtered to obtain a suspension having a desired distribution of optimised microbubbles 3. The blood clotting agents may be suspended or dissolved in a liquid layer of the shell. This may be the same layer in which the magnetic nanoparticles are suspended or an alternative layer. It may be necessary to modify the therapeutic agent for suspension or dissolution in a layer of the shell, but this may be achieved using methods known in the art. Alternatively, the blood clotting agent may be attached to, or incorporated in, the external coating of the microbubble using methods known in the art.

Recent studies that have investigated the use of magnetic microbubbles have thus far only used simple capillary flow models. This consists of an acoustically transparent tubing material encased within an agar based mixture. Agar is a tissue-mimicking material and as it is cast around the tubing a cylindrical cavity is left allowing for flow through the phantom. However a pseudo aneurysm is not a uniform structure but a perturbed non-homogeneous structure. The experimental challenge was to design a method of creating this, relatively complicated, cavity shape inside the agar cast. To solve this, the inventors used rapid prototyping (RP) to design an anthropomorphic flow phantom for ultrasound imaging. The inventors fabricated a hollow diseased carotid bifurcation model and cast the phantom around the model. The vessels where coupled to the surrounding agar hence creating a thin-walled flow phantom. There was a slight acoustic mismatch between the thin-walled structure and the cast, however demonstrated the potential of RP to fabricate complex vascular systems allowing for further in-vitro research.

This investigation designed a novel method of RP based phantom fabrication. It utilized the inherent feature of agar, consisting of almost entirely water, to dissolve the 3D printed model material within the casting.

This results in a complex cavity structure within the agar mould that can be used to investigate flow characteristics in anthropomorphic vascular systems. The pseudo aneurysm model was built using a soluble polyvinyl alcohol (PVA) filament 1.75 mm, RepRap Central, Sussex, United Kingdom) and printed using the MakerBot Replicator 2× (MakerBot, New York, USA). A simplified model of a pseudo aneurysm is shown in FIG. 1, however due to time constraints and the challenges regarding PVA printing the prototyped model was simplified even further It was simplified to a large sphere (sack) placed on top of a small cylinder (neck) protruding from a long cylinder (artery). FIG. 11 is an image of the model used throughout the investigation.

The dimensions for the model shown in FIG. 11 were set to; Rₛ=20 mm, xₙ=10 mm, Dₙ=5 mm and the diameter of the ‘artery’ as 8 mm. These values were provided by a clinical expert at the Nuffield Department of Surgical Sciences at the University of Oxford.

FIG. 10A is a schematic representation of the experimental setup used throughout the investigation. The flow and its value were created using a series of latex tubes and a high precision syringe pump (Harvard Apparatus PHD 2000 Infuse/Withdraw pump). An Ultrasound linear array transducer (9.4 MHz LA523, Esaote, Italy) was positioned above and coupled to the agar phantom using ANAGEL ultrasound transmission gel (AnaWiz Ltd, Surrey, UK). Video sequences were acquired using a ULA-OP ultrasound system (Mircoelectronic Systems Design Laboratory, Universita degli Studi di Firenze, Florence, Italy) at a pulse repetition frequency of 8 kHz. The ULA-OP system was used in B-mode ultrasound which is the standard method of ultrasound imaging.

An in-house machined cylindrical permanent magnet stack was placed above the phantom to act as the external magnetic field. The magnet was optimized to confine magnetic fields in a well defined spatial volume, the sphere/sack. To avoid downstream leakage the microbubbles where injected directly into the sack, similar to the current medical practice.

To represent a clinically relevant model, the flow rate used throughout the experiment was set to 0.22 ml/s.

Two separate experiments were carried out; one with and one without the magnetic effects. This was to investigate whether the external magnetic field could successfully retain the bubbles for longer periods of time. To quantify this, an image was taken at the point of microbubble injection and continually sampled for 120 s afterwards.

To further quantify the results, image processing was carried out using Matlab (2014B, TheMathsWorks, Natick, Mass., USA). It is well documented that ultrasound image intensity is directly proportional to microbubble concentration for clinically relevant values and therefore can be used as an indicator of the quantity of bubbles retained. To normalize the analysis a percentage difference of a specific region of interest was taken between an original reference image and a continuous series of images after the bubbles were injected. This was to quantify the results, as the percentage difference is expected to be larger for the magnetically targeted bubbles.

The novel method of complex flow phantom design resulted in successful cavity structures being created within the agar cast. The confined PVA model held within the agar eventually dissolved and was easily rinsed out of the phantom.

FIG. 12 is two images of the long cylinder, representing the artery, from an elevation and end elevation viewpoint. FIG. 12a depicts the length of the artery and also depicts how the soft PVA model bent during the casting. This bending was theorized to be due to the buoyancy of the PVA model and had little effect on the flow dynamics. FIG. 12b depicts how the structure is hollow and will allow for flow through the phantom. This is a clear indication of the potential of this non-invasive phantom design method and was used effectively throughout the experimental sections of this investigation.

FIG. 10C is an image of the sack structure taken directly before the microbubble injection. The setup seen in FIG. 10A was used to capture the image. Also depicted is a region of interest (ROI) used in the percentage difference calculation. It is clear from this image that there is a low average intensity inside the sack due to the lack of contrast agent.

FIG. 10 B is an image taken 100 s post-microbubble injection and clearly depicts an increase in the pixel intensities. This due to the presence of a contrast agent in the flow model. It is also clear that the entire sack structure was filled with microbubbles and a circular flow around the structure was observed. There is also a large dark streak clearly visible through the structure to which the origin of this artefact is unknown and requires further investigation.

FIG. 10D is a graphical representation of the microbubble retention rates within the rectangular ROI shown in FIGS. 10B and 10C. Depicted are the results for both magnetic targeting and non-magnetic targeting over a time of ˜120 s. The y-axis represents the percentage difference in pixel intensities of the resulting images with an original reference image. The x-axis represents the total number of frames taken during the video sequencing. It is clear that at 25 s (frame number 2000), after recording, the microbubbles were injected as there is a clear large spike in the percentage difference.

The figure also clearly shows that with the presence of a magnetic field the rate at which the microbubbles disperse is a lot slower than with no magnetic held presence. In the presence of a magnetic field and after 120 s there is a 251.16%±0.05% difference in pixel intensity between the final image taken and the original reference image. For non-magnetically targeted bubbles, there is ˜0% after 120 s. This indicates that under the influence of a magnetic field the microbubbles are retained for longer inside the sack. This is due to the magnetic forces acting on the bubbles due to the field and act as a trapping mechanism against the system flow.

FIGS. 10B and 10C depict the sack before and 100 s after microbubble injection. FIG. 10D illustrates the percentage difference in average pixel intensity for a region of interest seen in FIGS. 10B and 10C. The percentage difference of magnetically targeted and non-magnetically targeted microbubbles where plotted in conjunction on FIG. 10D. This clearly demonstrates that the rate of microbubble dispersion is much larger for non-magnetically targeted microbubbles and shows how an external magnetic field can be used to trap bubbles to a specific target area. After 120 s the percentage difference of microbubbles under a magnetic field was 251.16%±0.05% compared to ˜0% for non-magnetically targeted bubbles. This investigation was a successful proof of concept and investigated the substantial potential of magnetic microbubbles as localized drug delivery agents for the treatment of pseudo aneurysms.

It will be understood that variations of the above described examples and embodiments are possible within the scope of the invention as defined by the appended claims.

1. A composition for use in the treatment of a pseudo aneurysm in a subject, said composition comprising
i) microbubbles;
ii) a magnetic material; and
iii) a blood clotting agent.

2. A composition for use in the treatment of a pseudo aneurysm in a subject, said composition comprising
i) microbubbles;
ii) a magnetic material; and
iii) a blood clotting agent;
said treatment comprising administering said composition directly into the pseudo aneurysm and applying a magnetic field to the pseudo aneurysm so as to retain the blood clotting agent within the pseudo aneurysm for a prolonged time period compared to a case where no magnetic field is applied to the pseudo aneurysm.

3. A composition for use according to claim 2, wherein the magnetic field has a field gradient configured to act on the magnetic material to prevent the magnetic material leaving the pseudo aneurysm.

4. A composition for use according to claim 2, wherein the magnetic field is configured such that a magnitude of magnetic force exerted on the magnetic material of the composition varies along an axis extending through a source of the magnetic filed and the magnetic force has a peak located a finite distance from the source of the magnetic field on the axis and within a target volume.

5. A composition for use according to claim 1, wherein the magnetic material of the composition is ferromagnetic; and/or, wherein the magnetic material of the composition comprises magnetic nanoparticles; and/or wherein the magnetic material of the composition comprises nanoparticles comprising, as a main component, Fe₃O₄ and/or Fe₂O₃; and/or, wherein the magnetic material of the composition comprises Janus nanoparticles; and/or wherein the magnetic material of the composition comprises nanoparticles having an average particle size of from 5 to 30 nm, and/or or the magnetic material of the composition comprises nanoparticles coated with a biocompatible coating.

6-10. (canceled)

11. A composition for use according to claim 1, wherein the blood clotting agent is selected from:
i) one or more of blood clotting factors I through XIII, for example thrombin;
ii) desmopressin, vasopressin, vitamin K, collagen, oxidized cellulose, gelatin, chitosan, alginic acid; and
iii) astringents such as tannic acid and vasoconstrictors such as epinephrine and angiotensin II;
and pharmaceutically acceptable salts, derivatives or prodrugs thereof, and combinations thereof.

12. A composition for use according to claim 1, wherein the blood clotting agent is selected from thrombin and fibrin, including pharmaceutically acceptable salts, derivatives and prodrugs thereof.

13. A composition for use according to claim 1, wherein the blood clotting agent is thrombin.

14. A composition for use according to claim 1, wherein the microbubble has a gas core comprising air, nitrogen, carbon dioxide, oxygen, a noble gas, or a perfluorocarbon gas or any mixture thereof; and/or wherein the microbubbles comprise a shell having a liquid layer and the magnetic material is suspended in the liquid layer of the shell; and/or wherein the microbubbles comprise a shell wherein said shell comprises an external coating; and optionally wherein the external coating comprises a polymer, a surfactant or a lipid.

15-17. (canceled)

18. A composition for use according to claim 1, wherein (i) the blood clotting agent is attached to the microbubble but not directly conjugated to the magnetic material; or (ii) the blood clotting agent is conjugated to the magnetic material.

19. A composition for use according to claim 1, wherein:
i) the magnetic material is an oleic acid or lipid coated nanoparticle and the blood clotting agent is coupled to the microbubble and/or the nanoparticle via a disulphide bond or a maleimide linker; or
ii) the magnetic material is a nanoparticle coated with albumin or another protein and the blood clotting agent is attached to the nanoparticle by physical adsorption; or
iii) the nanoparticle is coated in avidin, streptavidin or neutravidin and the blood clotting agent is conjugated to biotin such that the blood clotting agent is attached to the nanoparticle by interaction of the avidin/streptavidin/neutravidin and the biotin; or
iv) the magnetic material comprises nanoparticles suspended in the liquid layer of the shell of the microbubble and the blood clotting agent is encapsulated in a liposome or is attached to or encapsulated in a polymeric particle, such that the liposome or polymeric particle can be coupled to the bubble surface either electrostatically or chemically.

20. A composition for use according to claim 11, wherein the nanoparticle is an iron oxide nanoparticle or a Janus nanoparticle and the blood clotting agent is thrombin or a pharmaceutically acceptable salt, derivative or prodrug thereof.

21. A composition for use according to claim 1, wherein the composition is parenterally administered directly into the pseudo aneurysm.

22. A composition for use according to claim 1, wherein the pseudo aneurysm is due to femoral artery puncture during cardiac catheterisation.

23. A composition for use according to claim 1, wherein the subject has an arteriovenous fistula.

24. A composition for use according to claim 1, wherein the step of administering the composition is performed with the guidance of ultrasound imaging, and/or wherein the treatment further comprises rupturing the microbubbles using ultrasound; and optionally wherein the imaging ultrasound and the microbubble rupturing ultrasound are provided by a single ultrasound transducer.

25-26. (canceled)

27. A method of imaging a pseudo aneurysm comprising:
applying a magnetic field to the pseudo aneurysm so as to retain within the pseudo aneurysm for a prolonged time period compared to a case where no magnetic field is applied to the pseudo aneurysm microbubbles comprising a magnetic material and a blood clotting agent that have been pre-administered directly into the pseudo aneurysm;
imaging the pseudo aneurysm using ultrasound, the microbubbles providing a contrast agent.

28. The method of claim 17 wherein the microbubbles comprising a magnetic material and a blood clotting agent that have been pre-administered directly into the pseudo aneurysm are comprised in a composition comprising: i) microbubbles; ii) a magnetic material; and iii) a blood clotting agent.

29. A method for treating a pseudo aneurysm in a subject, said method comprising administering to said subject a composition comprising:
i) microbubbles;
ii) a magnetic material; and
iii) a blood clotting agent;
said method comprising administering said composition directly into the pseudo aneurysm and applying a magnetic field to the pseudo aneurysm so as to retain the blood clotting agent within the pseudo aneurysm for a prolonged time period compared to a case where no magnetic field is applied to the pseudo aneurysm.

30. A composition comprising
i) microbubbles;
ii) a magnetic material; and
iii) a blood clotting agent;
for use in the manufacture of a medicament for the treatment of a pseudo aneurysm in a subject, said treatment comprising administering said medicament directly into the pseudo aneurysm and applying a magnetic field to the pseudo aneurysm so as to retain the blood clotting agent within the pseudo aneurysm for a prolonged time period compared to a case where no magnetic field is applied to the pseudo aneurysm.
